SEQUENCE LISTING (10 sequences)

SEQ ID NO: 1 - DNA, 20 bp, Homo sapiens
tgctctcctgttgtgcttct

SEQ ID NO: 2 - DNA, 20 bp, Homo sapiens
atagatggtcaatgcggcgt

SEQ ID NO: 3 - DNA, 20 bp, Influenza A virus
atcggaacttctggaggggt

SEQ ID NO: 4 - DNA, 20 bp, Influenza A virus
caggacttgtgagcaaccga

SEQ ID NO: 5 - DNA, 20 bp, Homo sapiens
gggagccaaaagggtcatca

SEQ ID NO: 6 - DNA, 20 bp, Homo sapiens
agtgatggcatggactgtgg

SEQ ID NO: 7 - protein, 1490 aa, Homo sapiens
MAGNSLVLPIVLWGRKAPTHCISAVLLTDDGATIVTGCHDGQICLWDLSVELQINPRALLFGHTASITCLSKACASSDKQYIVSASESGEMCLWDVSDGRCIEFTKLACTHTGIQFYQFSVGNQREGRLLCHGHYPEILVVDATSLEVLYSLVSKISPDWISSMSIIRSHRTQEDTVVALSVTGILKVWIVTSEISDMQDTEPIFEEESKPIYCQNCQSISFCAFTQRSLLVVCSKYWRVFDAGDYSLLCSGPSENGQTWTGGDFVSSDKVIIWTENGQSYIYKLPASCLPASDSFRSDVGKAVENLIPPVQHILLDRKDKELLICPPVTRFFYGCREYFHKLLIQGDSSGRLNIWNISDTADKQGSEEGLAMTTSISLQEAFDKLNPCPAGIIDQLSVIPNSNEPLKVTASVYIPAHGRLVCGREDGSIVIVPATQTAIVQLLQGEHMLRRGWPPHRTLRGHRNKVTCLLYPHQVSARYDQRYLISGGVDFSVIIWDIFSGEMKHIFCVHGGEITQLLVPPENCSARVQHCICSVASDHSVGLLSLREKKCIMLASRHLFPIQVIKWRPSDDYLVVGCSDGSVYVWQMDTGALDRCVMGITAVEILNACDEAVPAAVDSLSHPAVNLKQAMTRRSLAALKNMAHHKLQTLATNLLASEASDKGNLPKYSHNSLMVQAIKTNLTDPDIHVLFFDVEALIIQLLTEEASRPNTALISPENLQKASGSSDKGGSFLTGKRAAVLFQQVKETIKENIKEHLLDDEEEDEEIMRQRREESDPEYRSSKSKPLTLLEYNLTMDTAKLFMSCLHAWGLNEVLDEVCLDRLGMLKPHCTVSFGLLSRGGHMSLMLPGYNQPACKLSHGKTEVGRKLPASEGVGKGTYGVSRAVTTQHLLSIISLANTLMSMTNATFIGDHMKKGPTRPPRPSTPDLSKARGSPPTSSNIVQGQIKQVAAPVVSARSDADHSGSDPPSAPALHTCFLVNEGWSQLAAMHCVMLPDLLGLDKFRPPLLEMLARRWQDRCLEVREAAQALLLAELRRIEQAGRKEAIDAWAPYLPQYIDHVISPGVTSEAAQTITTAPDASGPEAKVQEEEHDLVDDDITTGCLSSVPQMKKISTSYEERRKQATAIVLLGVIGAEFGAEIEPPKLLTRPRSSSQIPEGFGLTSGGSNYSLARHTCKALTFLLLQPPSPKLPPHSTIRRTAIDLIGRGFTVWEPYMDVSAVLMGLLELCADAEKQLANITMGLPLSPAADSARSARHALSLIATARPPAFITTIAKEVHRHTALAANTQSQQNMHTTTLARAKGEILRVIEILIEKMPTDVVDLLVEVMDIIMYCLEGSLVKKKGLQECFPAICRFYMVSYYERNHRIAVGARHGSVALYDIRTGKCQTIHGHKGPITAVAFAPDGRYLATYSNTDSHISFWQMNTSLLGSIGMLNSAPQLRCIKTYQVPPVQPASPGSHNALKLARLIWTSNRNVILMAHDGKEHRFMV

SEQ ID NO: 8 - protein, 180 aa, Homo sapiens
MAALDLRAELDSLVLQLLGDLEELEGKRTVLNARVEEGWLSLAKARYAMGAKSVGPLQYASHMEPQVCLHASEAQEGLQKFKVVRAGVHAPEEVGPREAGLRRRKGPTKTPEPESSEAPQDPLNWFGILVPHSLRQAQASFRDGLQLAADIASLQNRIDWGRSQLRGLQEKLKQLEPGAA

SEQ ID NO: 9 - protein, 208 aa, Homo sapiens
MASSLLAGERLVRALGPGGELEPERLPRKLRAELEAALGKKHKGGDSSSGPQRLVSFRLIRDLHQHLRERDSKLYLHELLEGSEIYLPEVVKPPRNPELVARLEKIKIQLANEEYKRITRNVTCQDTRHGGTLSDLGKQVRSLKALVITIFNFIVTVVAAFVCTYLGSQYIFTEMASRVLAALIVASVVGLAELYVMVRAMEGELGEL

SEQ ID NO: 10 - protein, 835 aa, Homo sapiens
MKRRTDPECTAPIKKQKKRVAELALSLSSTSDDEPPSSVSHGAKASTTSLSGSDSETEGKQHSSDSFDDAFKADSLVEGTSSRYSMYNSVSQKLMAKMGFREGEGLGKYSQGRKDIVEASSQKGRRGLGLTLRGFDQELNVDWRDEPEPSACEQVSWFPECTTEIPDTQEMSDWMVVGKRKMIIEDETEFCGEELLHSVLQCKSVFDVLDGEEMRRARTRANPYEMIRGVFFLNRAAMKMANMDFVFDRMFTNPRDSYGKPLVKDREAELLYFADVCAGPGGFSEYVLWRKKWHAKGFGMTLKGPNDFKLEDFYSASSELFEPYYGEGGIDGDGDITRPENISAFRNFVLDNTDRKGVHFLMADGGFSVEGQENLQEILSKQLLLCQFLMALSIVRTGGHFICKTFDLFTPFSVGLVYLLYCCFERVCLFKPITSRPANSERYVVCKGLKVGIDDVRDYLFAVNIKLNQLRNTDSDVNLVVPLEVIKGDHEFTDYMIRSNESHCSLQIKALAKIHAFVQDTTLSEPRQAEIRKECLRLWGIPDQARVAPSSSDPKSKFFELIQGTEIDIFSYKPTLLTSKTLEKIRPVFDYRCMVSGSEQKFLIGLGKSQIYTWDGRQSDRWIKLDLKTELPRDTLLSVEIVHELKGEGKAQRKISAIHILDVLVLNGTDVREQHFNQRIQLAEKFVKAVSKPSRPDMNPIRVKEVYRLEEMEKIFVRLEMKIIKGSSGTPKLSYTGRDDRHFVPMGLYIVRTVNEPWTMGFSKSFKKKFFYNKKTKDSTFDLPADSIAPFHICYYGRLFWEWGDGIRVHDSQKPQDQDKLSKEDVLSFIQMHRA

(19) United States
(12) Patent Application Publication
(10) Pub. No.: US 2022/0002400 A1
(43) Pub. Date: Jan. 6, 2022

(54) Methods of Treating Influenza A Virus Infections

(71) Applicants: President and Fellows of Harvard College, Cambridge, MA (US); The General Hospital Corporation dba Massachusetts General Hospital, Boston, MA (US); University of Edinburgh, Midlothian (GB)

(72) Inventors: Bo Li, Cambridge, MA (US); Nir Hacohen, Boston, MA (US); J. Kenneth Baillie, Midlothian (GB)

(21) Appl. No.: 17/484,995

(22) Filed: Sep. 24, 2021

Related U.S. Application Data
(63) Continuation of application No. PCT/US2020/024919, filed on Mar. 26, 2020.
(60) Provisional application No. 62/824,554, filed on Mar. 27, 2019.

(51) Int. Cl. C07K 16/28; C12N 15/113; C07K 16/18; C07K 16/40; A61P 31/16

(52) U.S. Cl. CPC C07K 16/28; C12N 15/113; C07K 16/18; C12N 15/1138; C07K 2317/76; C07K 16/40; A61P 31/16; C12N 2310/11; C12N 2310/14; C12N 15/1137

(57) ABSTRACT

Methods of treating influenza A are provided, comprising administering to a subject at least one agent selected from a WDR7 inhibitor, a CCDC115 inhibitor, a TMEM199 inhibitor, and a CMTR1 inhibitor.

### INCORPORATION BY REFERENCE TO ANY PRIORITY APPLICATIONS

The present application is a continuation of International Application No. PCT/US2020/024919, filed Mar. 26, 2020, which claims the benefit of priority to U.S. Provisional Patent Application No. 62/824,554, filed Mar. 27, 2019, which is hereby incorporated by reference in its entirety.

### STATEMENT OF GOVERNMENT FUNDING

This invention was made with government support under Grant No. HG006193 awarded by the National Institutes of Health. The government has certain rights in the invention.

### SEQUENCE LISTING

The present application is filed with a Sequence Listing in electronic format. The Sequence Listing is provided as a file entitled “2021-09-22_01180-0003-00US_Sequence_Listing_ST25” created on Sep. 22, 2021, which is 24,817 bytes in size. The information in the electronic format of the sequence listing is incorporated herein by reference in its entirety.

### FIELD

The present application relates to the field of treatment of influenza A virus infections.

### BACKGROUND

Influenza A virus (IAV) infection remains a major health issue in the world today. The 2009 H1N1 pandemic resulted in over 60 million infected cases in the United States and over 10,000 deaths (Shrestha S S, et al. Clin Infect Dis. 2011; 52 Suppl 1: S75-82). Avian Influenza strains like the H5N1 and H7N9 have also crossed the species barrier and caused lethal infections in humans in recent years, raising concerns for future pandemics. (Gao R, et al. N Engl J Med. 2013; 368(20): 1888-97; Webster R G, Govorkova E A. N Engl J Med. 2006; 355(21):2174-7; Yen H L, Webster R G. Curr Top Microbiol Immunol. 2009; 333; 3-24.) Although vaccination regimes against seasonal flu have been fairly effective, there is still a lack of therapeutic options for people who do become infected. Current treatments including the neuraminidase inhibitors (Zanamivir and Oseltamivir) and M2 channel blockers (Amantadine and Rimantadine) have limited efficacy and are becoming less relevant due to the rapid emergence of resistant virus strains. (Bright R A, et al. JAMA. 2006; 295(8):891-4; Dawood F S, et al. N Engl J Med. 2009; 360(25):2605-15; Nicoll A, et al. Euro Surveill. 2008; 13(5).)

Like most viruses, IAV has a relatively small genome and limited repertoire of encoded proteins, and as such relies on the host machinery to replicate and complete its life cycle. (Bouvier N M, Palese P. Vaccine. 2008; 26 Suppl 4:D49-53.) Identification of host dependency factors that are necessary for IAV replication thus provides an attractive strategy for discovering new therapeutic targets due to higher barrier against drug resistance. To achieve this end, seven large-scale RNA interference (RNAi) screens have been performed in recent years, reporting a total of 1,362 host dependency factors that are important for IAV replication. (Brass A L, et al. Cell; 2009. 139(7): 1243-54; Hao L, et al. Nature. 2008; 14; 454(7206): 890-3; Karlas A, et al. Nature. 2010; 463(7282):818-22; Konig R, et al. Nature. 2010; 463(7282):813-7; Shapira S D, et al. Cell. 2009; 139(7):1255-67; Sui B, et al. Virology. 2009; 387(2):473-8; Tran A T, et al. Cell Death Dis. 2013; 4:e746.) However, the number of overlapping hits between these studies has been minimal, with only 6 hits being found in at least four screens and none in all seven. (Chou Y C, et al. J Clin Bioinforma. 2015; 5:2.) This indicates a high false positive rate which may be attributed to both differences in the screen systems and intrinsic limitations of RNAi. (Dong H, et al. Antiviral Res. 2008; 80(1):1-10.) This is also consistent with studies aimed at identifying host factors for HIV infection, where there is poor overlap between three independent screens¹⁷⁻¹⁹. (Konig R, et al. Cell. 2008; 135:49-60; Brass A L, et al. Science. 2008; 319:921-926; Zhou H, et al. Cell Host Microbe. 2008; 4:495-504.)

The past decade has witnessed a number of deadly Influenza outbreaks which calls for the need of new and effective antiviral therapy.

### SUMMARY

In various embodiments, methods of treating influenza A virus infection are provided, comprising administering to a subject in need thereof an effective amount of at least one agent selected from a WDR7 inhibitor, a CCDC115 inhibitor, a TMEM199 inhibitor, and a CMTR1 inhibitor.

In some embodiments, at least one agent is a WDR7 inhibitor. In some embodiments, the WDR7 inhibitor is selected from an antibody, an antisense oligonucleotide, an siRNA, a peptide, an abzyme, or a small molecule. In some embodiments, the WDR7 inhibitor is a small molecule. In some embodiments, the WDR7 inhibitor is an antisense oligonucleotide or an siRNA. In some embodiments, the antisense oligonucleotide is complementary to a portion of the WDR7 mRNA. In some embodiments, the WDR7 inhibitor is a peptide. In some embodiments, the WDR7 inhibitor is an antibody. In some embodiments, the antibody is an antibody fragment. In some embodiments, the antibody fragment is selected from an scFv, Fab, Fab′, F(ab′)2 fragment.

In some embodiments, at least one agent is a CCDC115 inhibitor. In some embodiments, the CCDC115 inhibitor is selected from an antibody, an antisense oligonucleotide, an siRNA, a peptide, an abzyme, or a small molecule. In some embodiments, the CCDC115 inhibitor is a small molecule. In some embodiments, the CCDC115 inhibitor is an antisense oligonucleotide or an siRNA. In some embodiments, the antisense oligonucleotide is complementary to a portion of the CCDC115 mRNA. In some embodiments, the CCDC115 inhibitor is a peptide. In some embodiments, the CCDC115 inhibitor is an antibody. In some embodiments, the antibody is an antibody fragment. In some embodiments, the antibody fragment is selected from an scFv, Fab, Fab′, F(ab′)2 fragment.

In some embodiments, at least one agent is a TMEM199 inhibitor. In some embodiments, the TMEM199 inhibitor is selected from an antibody, an antisense oligonucleotide, an siRNA, a peptide, an abzyme, or a small molecule. In some embodiments, the TMEM199 inhibitor is a small molecule. In some embodiments, the TMEM199 inhibitor is an antisense oligonucleotide or an siRNA. In some embodiments, the antisense oligonucleotide is complementary to a portion of the TMEM199 mRNA. In some embodiments, the TMEM199 inhibitor is a peptide. In some embodiments, the TMEM199 inhibitor is an antibody. In some embodiments, the antibody is an antibody fragment. In some embodiments, the antibody fragment is selected from an scFv, Fab, Fab′, F(ab′)2 fragment.

In some embodiments, at least one agent is a CMTR1 inhibitor. In some embodiments, the CMTR1 inhibitor is selected from an antibody, an antisense oligonucleotide, an siRNA, a peptide, an abzyme, or a small molecule. In some embodiments, the CMTR1 inhibitor is a small molecule. In some embodiments, the CMTR1 inhibitor is an antisense oligonucleotide or an siRNA. In some embodiments, the antisense oligonucleotide is complementary to a portion of the CMTR1 mRNA. In some embodiments, the CMTR1 inhibitor is a peptide. In some embodiments, the CMTR1 inhibitor is an antibody. In some embodiments, the antibody is an antibody fragment. In some embodiments, the antibody fragment is selected from an scFv, Fab, Fab′, F(ab′)2 fragment.

In some embodiments, the agent reduces nuclear entry of influenza A. In some embodiments, the agent reduces viral replication. In some such embodiments, the agent is selected from a WDR7 inhibitor, a CCDC115 inhibitor, and a TMEM199 inhibitor. In some embodiments, the agent reduces viral transcription. In some such embodiments, the agent is a CMTR1 inhibitor.

In some embodiments, the influenza A is selected from H1N1, H3N2, H5N1, and H7N9 influenza A. In some embodiments, the influenza A is selected from H1N1 and H3N2 influenza A.

In some embodiments, the subject is suspected of having an influenza A virus infection. In some embodiments, the subject is at risk of developing an influenza A virus infection. In some embodiments, the subject has been diagnosed with an influenza A virus infection. In some embodiments, the subject exhibits at least one symptom of influenza A virus infection. In some embodiments, at least one symptom is selected from fever, muscle ache, chills, headache, cough, fatigue, nasal congestion, and sore throat. In some embodiments, treating influenza A virus infection comprises reducing the severity and/or duration of one or more symptoms of influenza A virus infection.

In some embodiments, the method further comprises administering a therapeutic agent selected from baloxavir marboxil, oseltamivir, and zanamivir to the subject. In some embodiments, the method comprises administering a CMTR1 inhibitor and baloxavir marboxil to the subject.

It is to be understood that both the foregoing general description and the following detailed description are exemplary and explanatory only and are not restrictive of the claims.

### BRIEF DESCRIPTION OF THE DRAWINGS

FIGS. 1A-1E. (A) Schematics of the genome-wide CRISPR screen strategy. (B) Log 2-fold change in abundance of sgRNA for genes enriched in HA-low bin compared to HA-high bin and −log 10 p-value of screen hits based on ranking. (C) Read count of individual sgRNAs enriched in HA-low bin (P6) versus HA-high bin (p4) for secondary screen. (D) Performance of integrative cross-validation (computed without the CRISPR data) and composites of input datasets against an unseen gold standard (CRISPR screen). Plot shows number of overlaps with CRISPR screen for a given position in each ranked dataset. In categories containing more than one data source, the composite cross-validation score within that category is used to create a composite ranked list. (E) Representative of shared information content between each data source after integrative cross-validation. Size of data source blocks is proportional to the summed information content (cross-validation scores) of input list. Lines are colored according to the dominant data source (i.e. the data source with the greater summed cross-validation scores for the set of overlapping genes between a given pair of data source).

FIG. 2. Flow cytometry of surface Hemagglutinin (HA) expression in CMAS-null, SLC35A1-null and wild type A549 cells 16 hours post-infection by PR8 virus at MOI5.

FIG. 3. Diagram showing hits from the CRISPR screen relative to the Influenza life cycle.

FIGS. 4A-4H. (A) Flow cytometry of surface HA expression in PR8-infected A549 cells transduced with either gene-specific or non-targeting sgRNAs. Table shows difference in percentage of HA+ cells for different knockout versus control cells. (B) Plaque assay measuring virus titer in plaque forming units (PFU)/ml. Supernatant was collected from Knockout and WT cells infected with PR8 at MOI 0.1 for 48 hours. Error bars represent standard deviations of three experimental replicates. (C) Plaque assay measuring virus titer. Cells were infected with UDORN virus at MOI 0.1 for 48 hours. (D) Flow cytometry of surface HA expression in PR8-infected Human lung fibroblasts (HLF) cells transduced with gene-specific or non-targeting sgRNAs. Transduced cells were sorted based on GFP expression. (E) Chart shows percentage of HA+ knockout cells normalized to that of control cells. (F) Proliferation curve of knockout cells versus control cells up to 9 days post-transduction. Error bars represent standard deviations of three experimental replicates. (G) ALAMAR blue assay. (H) Annexin V staining of A549 cells on day 9 post-transduction with gene-specific or non-targeting sgRNAs.

FIGS. 5A-5C. Analysis of genomic editing efficiency by massively parallel sequencing of the CRISPR target site. Charts show (A) high inhibition, (B) moderate inhibition, and (C) low inhibition. All Knockout cells have high frequency of INDEL (>95%).

FIG. 6. Bar graph showing percentage of HA+A549 cells 16 hours post-infection with PR8 virus. Cells were either transduced with sgRNA alone or together with a rescue plasmid containing a codon-mutated version of the gene. Percentage values are normalized to that of control cells.

FIGS. 7A-7H. (A) Flow cytometry and Fluorescent-In-Situ-Hybridization (FISH) for PR8 NP protein (top) and RNA levels (bottom) at 4 hours post-infection. (B) Bar graph shows quantification for the percentage of NP+ cells both protein. (C) Bar graph shows quantification for the percentage of NP+ cells for RNA. Error bars represent standard deviation from three experimental replicates. (D) Flow cytometry of GFP expression in knockout versus control A549 cells that have been transduced with a MLV-GFP retrovirus pseudotyped with H1N1 IAV HA and NA envelop proteins. Bar graphs show quantification of percentage of GFP+ cells that were transduced either with HA/NA pseudotyped virus (E) or MLV-Env pseudotyped virus (F). (G) Flow cytometry of surface bound HA after knockout and control cells were incubated with PR8 virus for 30 minutes at 4° C. (H) Flow cytometry of surface sialic acid level. Cells were stained with fluorescein-labelled Sambucus nigra Lectin for 1 hour at 4° C.

FIGS. 8A-8F. (A) Immunofluorescent staining of knockout and control cells with Lysotracker red, anti-LAMP1 and anti-Rab7 antibodies on day 9 post-sgRNA transduction. Cells were incubated with Lysotracker red for 1 hour at 37° C., followed by fixation and staining with fluorophore-conjugated anti-LAMP1 and anti-Rab7 antibodies at 4° C. overnight. (B) Lysotracker red and Lysosensor blue staining of knockout and control cells on day 9 post-sgRNA transduction. Cells were incubated with the both dyes for 1 hour at 37° C. followed by fixation. (C) Bar graph shows quantification for lysotracker red staining. (D) Bar graph shows quantification for lysotracker blue staining. (E) Lysotracker red staining of knockout and control cells on day 9 post-sgRNA transduction. Cells were either mock-treated or treated with 100 nM Bafilomycin A (BafA) for 1 hours at 37° C. prior to Lysotracker red staining. (F) Bar graph showing percentage of HA+ cells 16 hours post-infection by PR8 virus. A549 cells were transduced with gene-specific or non-targeting sgRNA for 9 days. Prior to PR8 infection, knockout and control cells were either mock-treated or treated with 100 nM Bafilomycin A (BafA) for 1 hours at 37° C. Percentage values are normalized to that of control cells. Error bars represent standard deviation from three experimental replicates.

FIGS. 9A-9B. (A) Lysotracker red staining of Human Lung Fibroblasts (HLF) on day 9 post-transduction with gene-specific or non-targeting sgRNA. (B) Bar graph shows quantification for lysotracker red staining.

FIG. 10. Lysotracker red staining of A549 cells that were transduced with lentivirus carrying empty vector or vectors expressing WDR7, CCDC115 or TMEM199. Cells were fixed and stained with Lysotracker red on day 7 post-transduction.

FIG. 11. Bar graph showing percentage of HA+A549 cells 16 hours post-infection with PR8 virus. Cells were either transduced with sgRNA alone or sgRNA+rescue plasmid expressing a codon-mutated version of the knockout gene. Percentage values are normalized to that of control cells (non-targeting sgRNA). Error bars represent standard deviation of three experimental replicates.

FIG. 12. Lysotracker red staining of knockout and rescue cells on day 9 post-sgRNA transduction. Cells were either transduced with sgRNA alone or together with a rescue plasmid containing codon-mutated version of the gene.

FIG. 13. Percentage of HA+A549 cells 16 hours post-infection with PR8 virus. Cells were transduced with gene-specific or non-targeting sgRNA for 9 days. They were then pre-treated with different concentrations of Bafilomycin A for 1 hour prior to PR8 infection.

FIG. 14. Bar graph showing percentage of HA+A549 cells 16 hours post-infection with PR8 virus. Cells were either transduced with sgRNA alone, sgRNA+rescue plasmid expressing a codon-mutated version of the knockout gene, or sgRNA+rescue plasmid encoding a different gene. Percentage values are normalized to that of control cells (non-targeting sgRNA). Error bars represent standard deviation of three experimental replicates.

FIGS. 15A-15D. (A) Immunofluorescent staining of knockout and control cells with Alexafluor647-conjugated transferrin and FITC Anti-transferrin receptor antibody. Cells were incubated with transferrin for 1 hours at 37° C. before fixation. (B) Immunofluorescent staining of knockout and control cells with FITC-conjugated anti-H1N1 NP antibody. Cells were infected with PR8 virus at M01200 for 2 hours at 37° C. (C) Fold change in viral NP RNA measured by qRT-PCR at different time points post-infection by PR8 virus. Fold change is normalized to viral RNA level at 10 min post-infection. Cells were either mocked treated or treated with 40 uM Importazole for 1 hour to inhibit nuclear import of IAV. (D) Immunofluorescent staining of knockout and control cells with DQ-green Bovine serum albumin (BSA) and Lysotracker red. Cells were treated with 20 ug/ml DQ-BSA and Lysotracker red for 1 hour at 37° C. before fixation.

FIGS. 16A-16E. (A) Luciferase activity measured by luminescence reader. Knockout and control cells were transfected with IAV PR8 PA, PB1, PB2 and NP-expressing plasmids together with green-Renilla and Luciferase reporter plasmids for 24 hours. Readings are normalized to that of control cells. Error bars represent standard deviation from three experimental replicates. (B) Fold change in IFN-β mRNA levels measured by qRT-PCR. A549 cells were transduced with gene-specific or non-targeting sgRNA for 9 days and infected with PR8 virus at M015. RNA is extracted 16 hours post-infection. Readings are normalized to that of control cells. Error bars represent standard deviation from three experimental replicates. (C) PCA analysis of RNA sequencing data from PR8-infected knockout and control cells. Table on the rights shows number of differentially expressed genes between different conditions. Each data represents an independent experimental replicate. (D) GO enrichment analysis showing top 10 up-regulated gene categories in knockout versus control cells. (E) Fold change in IFN-β mRNA levels measured by qRT-PCR. CMTR1-KO cells were either transduced with sgRNA specific for RIG-I, MAV, IRF3, or non-targeting sgRNA for 9 days. They were then infected with PR8 virus at M015 and RNA is extracted 16 hours post-infection. Readings are normalized to that of control cells. Error bars represent standard deviation from three experimental replicates.

FIG. 17. Effect of Xofluza® (baloxavir marboxil) treatment on Influenza A/Puerto Rico/8/34 infection in sgRNA-transduced (WDR7−/−, CCDC115−/−, TMEM199−/−, and CMTR1−/−) and wild type cell lines. The Y-axis shows the percentage of HA+ cells normalized to untreated cells.

### DESCRIPTION OF CERTAIN EMBODIMENTS

### I. Definitions

As used herein, the term “about” refers to a numeric value, including, for example, whole numbers, fractions, and percentages, whether or not explicitly indicated. The term “about” generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. In some embodiments, an antibody may be a chimeric antibody, a humanized antibody, or a human antibody.

The term antibody includes, but is not limited to, fragments that are capable of binding to an antigen, such as Fv, single-chain Fv (scFv), Fab, Fab′, di-scFv, sdAb (single domain antibody) and (Fab′)₂ (including a chemically linked F(ab′)2). The term antibody also includes, but is not limited to, chimeric antibodies, humanized antibodies, and antibodies of various species such as mouse, human, cynomolgus monkey, etc. Antibody fragments also include either orientation of single chain scFvs, tandem di-scFv, diabodies, tandem tri-sdcFv, minibodies, etc. Antibody fragments also include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv).

An “abzyme” or “catalytic antibody” refers to a monoclonal antibody with catalytic activity.

The term “antisense oligonucleotide” refers to a single-stranded oligonucleotide comprising 8 to 50 monomeric units and having a nucleobase sequence that permits hybridization to a corresponding segment of a target nucleic acid. An antisense oligonucleotide may comprise natural, non-natural, and/or modified nucleosides and/or internucleoside linkages.

The term “siRNA” refers to a double-stranded oligonucleotide comprising a first strand comprising 10 to 30 monomeric units and a second strand comprising 10 to 30 monomeric units that is complementary to the first strand, wherein the first strand or second strand has a nucleobase sequence that permits hybridization to a corresponding segment of a target nucleic acid. The first strand and second strand may have 0, 1, 2, or 3 mismatches with respect to one another.

The term “monoclonal antibody” refers to an antibody of a substantially homogeneous population of antibodies, that is, the individual antibodies comprising the population are identical except for possible naturally-occurring mutations that may be present in minor amounts. Monoclonal antibodies are specific, being directed against a single antigenic site. Furthermore, in contrast to polyclonal antibody preparations, which typically include different antibodies directed against different determinants (epitopes), each antibody in a monoclonal antibody preparation is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method, by recombinant DNA methods, or be isolated from phage libraries.

The term “peptide” as used herein refers to a molecule formed by linking at least two, and up to 300, amino acids by amide bonds. The amino acids of a peptide may be natural, non-natural, and/or modified amino acids. In some embodiments, a peptide comprises 2-200 amino acids, or 2-100 amino acids, or 2-50 amino acids, or 2-30 amino acids, or 10-300 amino acids, or 10-200 amino acids, or 10-100 amino acids, or 10-50 amino acids.

The term “vector” is used to describe a polynucleotide that can be engineered to contain a cloned polynucleotide or polynucleotides that can be propagated in a host cell. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, cerebrospinal fluid, other cells, organs, and tissues.

A “reference” as used herein, refers to any sample, standard, or level that is used for comparison purposes. A reference may be obtained from a healthy and/or non-diseased sample. In some examples, a reference may be obtained from an untreated sample, or may be a sample from the subject prior to treatment. In some examples, a reference is obtained from one or more healthy individuals who are not the subject or patient.

“WD repeat domain 7” and “WDR7” as used herein refer to any native WDR7 that results from expression and processing of WDR7 in a cell. The term includes WDR7 from any vertebrate source, including mammals such as primates (e.g., humans and cynomolgus monkeys) and rodents (e.g., mice and rats), unless otherwise indicated. The term also includes naturally occurring variants of WDR7, e.g., splice variants, isoforms, isozymes, or allelic variants. The amino acid sequence of an exemplary human WDR7 protein is shown in SEQ ID NO: 7.

“Coiled-coil domain containing protein 115” and “CCDC115” as used herein refer to any native ADSS that results from expression and processing of CCDC115 in a cell. The term includes CCDC115 from any vertebrate source, including mammals such as primates (e.g., humans and cynomolgus monkeys) and rodents (e.g., mice and rats), unless otherwise indicated. The term also includes naturally occurring variants of CCDC115, e.g., splice variants, isoforms, isozymes, or allelic variants. The amino acid sequence of an exemplary human CCDC115 protein is shown in SEQ ID NO: 8.

“Transmembrane protein 199” and “TMEM199” as used herein refer to any native TMEM199 that results from expression and processing of TMEM199 in a cell. The term includes TMEM199 from any vertebrate source, including mammals such as primates (e.g., humans and cynomolgus monkeys) and rodents (e.g., mice and rats), unless otherwise indicated. The term also includes naturally occurring variants of TMEM199, e.g., splice variants, isoforms, isozymes, or allelic variants. The amino acid sequence of an exemplary human TMEM199 protein is shown in SEQ ID NO: 9.

“Cap methyltransferase 1” and “CMTRT” as used herein refer to any native CMTR1 that results from expression and processing of CMTR1 in a cell. The term includes CMTR1 from any vertebrate source, including mammals such as primates (e.g., humans and cynomolgus monkeys) and rodents (e.g., mice and rats), unless otherwise indicated. The term also includes naturally occurring variants of CMTR1, e.g., splice variants, isoforms, isozymes, or allelic variants. The amino acid sequence of an exemplary human CMTR1 protein is shown in SEQ ID NO: 10.

A “WDR7 inhibitor” or refers to an agent that inhibits the expression, activity, and/or level of WDR7.

A “CCDC115 inhibitor” or refers to an agent that inhibits the expression, activity, and/or level of CCDC115.

A “TMEM199 inhibitor” or refers to an agent that inhibits the expression, activity, and/or level of TMEM199.

A “CMTR1 inhibitor” or refers to an agent that inhibits the expression, activity, and/or level of CMTR1.

An “effective amount” of an agent, e.g., a pharmaceutical formulation, refers to an amount effective, at dosages and for periods of time necessary, to achieve the desired therapeutic or prophylactic result.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In various embodiments, the individual or subject is a human.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of a disease or condition, alleviation of symptoms, diminishment of any direct or indirect pathological consequences of the disease or condition, decreasing the rate of disease progression, amelioration or palliation of the disease or condition, and remission or improved prognosis. In some embodiments, methods are provided that delay development of a condition or disease or one or more symptoms of the condition or disease, or slow the progression of a disease or condition. In some embodiments, treatment comprises reducing the severity and/or duration of symptoms.

### II. Exemplary Therapeutic Methods

Methods of treating influenza A infection are provided. The present inventors have identified the V-type ATPase accessory proteins WDR7, CCDC115, and TMEM199 as involved in endo-lysosomal acidification, which is needed for nuclear entry of influenza A. Inhibition of WDR7, CCDC115, or TMEM199 disrupts the pH gradient from early to late endosomes, which IAV requires for entry into the nucleus, where viral replication takes place. The present inventors have also identified CMTR1, a 2′-O-ribose cap methyltransferase, as needed for IAV cap snatching, and thus in viral transcription.

Accordingly, in some embodiments, a method of treating influenza A comprises administering to a subject in need thereof an effective amount of at least one agent selected from a WDR7 inhibitor, a CCDC115 inhibitor, a TMEM199 inhibitor, and a CMTR1 inhibitor. In various embodiments, methods comprise administering to a subject having or suspected of having an influenza A infection at least one agent selected from a WDR7 inhibitor, a CCDC115 inhibitor, a TMEM199 inhibitor, and a CMTR1 inhibitor. In various embodiments, the subject has been identified as having an influenza A infection, or is suspected of having an influenza A infection, or is predicted to develop an influenza A infection, or is at risk for developing an influenza A infection.

In some embodiments, treating influenza A comprises reducing the severity and/or duration of at least one symptom of influenza A. In some embodiments, at least one symptom of influenza A is selected from fever, muscle ache, chills, headache, cough, fatigue, nasal congestion, and sore throat.

In various embodiments, the influenza A is selected from H1N1, H3N2, H5N1, and H7N9.

### A. Exemplary WDR7 Inhibitors

In some embodiments, a method of treating influenza A comprises administering to a subject in need thereof at least one WDR7 inhibitor. A WDR7 inhibitor refers to an agent that reduces the expression, activity, and/or level of WDR7. That is, in various embodiments, a WDR7 inhibitor may inhibit the expression of the WDR7 protein, e.g., by inhibiting translation of the WDR7 mRNA into the WDR7 protein. In some embodiments, a WDR7 inhibitor inhibits the activity of WDR7, such as by binding to WDR7 and interfering with its activity.

In various embodiments, a WDR7 inhibitor may be an antibody, an antisense oligonucleotide, an siRNA, a peptide, an abzyme, or a small molecule.

In some embodiments, a WDR7 inhibitor is a small molecule. A small molecule WDR7 inhibitor may, in some embodiments, bind to the WDR7 and inhibit its interaction with V-type ATPase.

In some embodiments, an WDR7 inhibitor is a peptide. A peptide is a polymeric compound of amino acids comprising up to 300 amino acid units linked by amide bonds. In some embodiments, a peptide inhibitor comprises fewer than 200, fewer than 100, fewer than 50, fewer than 40, fewer than 30, fewer than 20, or fewer than 10 amino acids. In some embodiments, a peptide inhibitor comprises 2-200 amino acids, or 2-100 amino acids, or 2-50 amino acids, or 2-30 amino acids, or 10-300 amino acids, or 10-200 amino acids, or 10-100 amino acids, or 10-50 amino acids. The amino acids of a peptide may be natural, non-natural, and/or modified.

In some embodiments, a WDR7 inhibitor is an antisense oligonucleotide. Antisense oligonucleotides are well known in the art. Antisense oligonucleotides are typically 8-50, 8-40, or 8-30 nucleosides long and, in some embodiments, comprise one or more modified nucleosides and/or modified base moieties and/or modified internucleoside linkages. In some embodiments, an antisense oligonucleotide mediates RNaseH activity, which causes degradation of the target mRNA. Antisense oligonucleotides are reviewed, for example, in Antisense Drug Technology, Ed. Stanley T. Corrke, CRC Press, 2007.

In some embodiments, a WDR7 inhibitor is an siRNA. siRNAs are double-stranded oligonucleotides in which one strand has a nucleobase sequence that permits hybridization to a corresponding segment of a target nucleic acid. siRNAs may comprise various modifications. Such modifications, and siRNAs generally, are well known in the art. See, e.g., siRNA Design: Methods and Protocols, Ed. Debra J. Taxman, Springer-Verlag New York, LLC, 2013.

In some embodiments, a WDR7 inhibitor is an antibody that binds WDR7 and inhibits one or more activities of WDR7. As noted herein, the term “antibody” includes various antibody structures, including but not limited to monoclonal antibodies, polyclonal antibodies, multispecific antibodies (e.g., bispecific antibodies), and antibody fragments so long as they exhibit the desired antigen-binding activity. In some embodiments, a WDR7 inhibitor is an abzyme.

### B. Exemplary CCDC115 Inhibitors

In some embodiments, a method of treating influenza A comprises administering to a subject in need thereof at least one CCDC115 inhibitor. A CCDC115 inhibitor refers to an agent that reduces the expression, activity, and/or level of CCDC115. That is, in various embodiments, a CCDC115 inhibitor may inhibit the expression of the CCDC115 protein, e.g., by inhibiting translation of the CCDC115 mRNA into the CCDC115 protein. In some embodiments, a CCDC115 inhibitor inhibits the activity of CCDC115, such as by binding to CCDC115 and interfering with its activity.

In various embodiments, a CCDC115 inhibitor may be an antibody, an antisense oligonucleotide, an siRNA, a peptide, an abzyme, or a small molecule.

In some embodiments, a CCDC115 inhibitor is a small molecule. A small molecule CCDC115 inhibitor may, in some embodiments, bind to the CCDC115 and inhibit its interaction with V-type ATPase.

In some embodiments, an CCDC115 inhibitor is a peptide. A peptide is a polymeric compound of amino acids comprising up to 300 amino acid units linked by amide bonds. In some embodiments, a peptide inhibitor comprises fewer than 200, fewer than 100, fewer than 50, fewer than 40, fewer than 30, fewer than 20, or fewer than 10 amino acids. In some embodiments, a peptide inhibitor comprises 2-200 amino acids, or 2-100 amino acids, or 2-50 amino acids, or 2-30 amino acids, or 10-300 amino acids, or 10-200 amino acids, or 10-100 amino acids, or 10-50 amino acids. The amino acids of a peptide may be natural, non-natural, and/or modified.

In some embodiments, a CCDC115 inhibitor is an antisense oligonucleotide. Antisense oligonucleotides are well known in the art. Antisense oligonucleotides are typically 8-50, 8-40, or 8-30 nucleosides long and, in some embodiments, comprise one or more modified nucleosides and/or modified base moieties and/or modified internucleoside linkages. In some embodiments, an antisense oligonucleotide mediates RNaseH activity, which causes degradation of the target mRNA. Antisense oligonucleotides are reviewed, for example, in Antisense Drug Technology, Ed. Stanley T. Corrke, CRC Press, 2007.

In some embodiments, a CCDC115 inhibitor is an siRNA. siRNAs are double-stranded oligonucleotides in which one strand has a nucleobase sequence that permits hybridization to a corresponding segment of a target nucleic acid. siRNAs may comprise various modifications. Such modifications, and siRNAs generally, are well known in the art. See, e.g., siRNA Design: Methods and Protocols, Ed. Debra J. Taxman, Springer-Verlag New York, LLC, 2013.

In some embodiments, a CCDC115 inhibitor is an antibody that binds CCDC115 and inhibits one or more activities of CCDC115. As noted herein, the term “antibody” includes various antibody structures, including but not limited to monoclonal antibodies, polyclonal antibodies, multispecific antibodies (e.g., bispecific antibodies), and antibody fragments so long as they exhibit the desired antigen-binding activity. In some embodiments, a CCDC115 inhibitor is an abzyme.

### C. Exemplary TMEM199 Inhibitors

In some embodiments, a method of treating influenza A comprises administering to a subject in need thereof at least one TMEM199 inhibitor. A TMEM199 inhibitor refers to an agent that reduces the expression, activity, and/or level of TMEM199. That is, in various embodiments, a TMEM199 inhibitor may inhibit the expression of the TMEM199 protein, e.g., by inhibiting translation of the TMEM199 mRNA into the TMEM199 protein. In some embodiments, a TMEM199 inhibitor inhibits the activity of TMEM199, such as by binding to TMEM199 and interfering with its activity.

In various embodiments, a TMEM199 inhibitor may be an antibody, an antisense oligonucleotide, an siRNA, a peptide, an abzyme, or a small molecule.

In some embodiments, a TMEM199 inhibitor is a small molecule. A small molecule TMEM199 inhibitor may, in some embodiments, bind to the TMEM199 and inhibit its interaction with V-type ATPase.

In some embodiments, an TMEM199 inhibitor is a peptide. A peptide is a polymeric compound of amino acids comprising up to 300 amino acid units linked by amide bonds. In some embodiments, a peptide inhibitor comprises fewer than 200, fewer than 100, fewer than 50, fewer than 40, fewer than 30, fewer than 20, or fewer than 10 amino acids. In some embodiments, a peptide inhibitor comprises 2-200 amino acids, or 2-100 amino acids, or 2-50 amino acids, or 2-30 amino acids, or 10-300 amino acids, or 10-200 amino acids, or 10-100 amino acids, or 10-50 amino acids. The amino acids of a peptide may be natural, non-natural, and/or modified.

In some embodiments, a TMEM199 inhibitor is an antisense oligonucleotide. Antisense oligonucleotides are well known in the art. Antisense oligonucleotides are typically 8-50, 8-40, or 8-30 nucleosides long and, in some embodiments, comprise one or more modified nucleosides and/or modified base moieties and/or modified internucleoside linkages. In some embodiments, an antisense oligonucleotide mediates RNaseH activity, which causes degradation of the target mRNA. Antisense oligonucleotides are reviewed, for example, in Antisense Drug Technology, Ed. Stanley T. Corrke, CRC Press, 2007.

In some embodiments, a TMEM199 inhibitor is an siRNA. siRNAs are double-stranded oligonucleotides in which one strand has a nucleobase sequence that permits hybridization to a corresponding segment of a target nucleic acid. siRNAs may comprise various modifications. Such modifications, and siRNAs generally, are well known in the art. See, e.g., siRNA Design: Methods and Protocols, Ed. Debra J. Taxman, Springer-Verlag New York, LLC, 2013.

In some embodiments, a TMEM199 inhibitor is an antibody that binds TMEM199 and inhibits one or more activities of TMEM199. As noted herein, the term “antibody” includes various antibody structures, including but not limited to monoclonal antibodies, polyclonal antibodies, multispecific antibodies (e.g., bispecific antibodies), and antibody fragments so long as they exhibit the desired antigen-binding activity. In some embodiments, a TMEM199 inhibitor is an abzyme.

### D. Exemplary CMTR1 Inhibitors

In some embodiments, a method of treating influenza A comprises administering to a subject in need thereof at least one CMTR1 inhibitor. A CMTR1 inhibitor refers to an agent that reduces the expression, activity, and/or level of CMTR1. That is, in various embodiments, a CMTR1 inhibitor may inhibit the expression of the CMTR1 protein, e.g., by inhibiting translation of the CMTR1 mRNA into the CMTR1 protein. In some embodiments, a CMTR1 inhibitor inhibits the activity of CMTR1, such as by binding to CMTR1 and interfering with its activity.

In various embodiments, a CMTR1 inhibitor may be an antibody, an antisense oligonucleotide, an siRNA, a peptide, an abzyme, or a small molecule.

In some embodiments, a CMTR1 inhibitor is a small molecule. A small molecule CMTR1 inhibitor may, in some embodiments, bind to the CMTR1 active site and inhibit its catalytic activity.

In some embodiments, an CMTR1 inhibitor is a peptide. A peptide is a polymeric compound of amino acids comprising up to 300 amino acid units linked by amide bonds. In some embodiments, a peptide inhibitor comprises fewer than 200, fewer than 100, fewer than 50, fewer than 40, fewer than 30, fewer than 20, or fewer than 10 amino acids. In some embodiments, a peptide inhibitor comprises 2-200 amino acids, or 2-100 amino acids, or 2-50 amino acids, or 2-30 amino acids, or 10-300 amino acids, or 10-200 amino acids, or 10-100 amino acids, or 10-50 amino acids. The amino acids of a peptide may be natural, non-natural, and/or modified.

In some embodiments, a CMTR1 inhibitor is an antisense oligonucleotide. Antisense oligonucleotides are well known in the art. Antisense oligonucleotides are typically 8-50, 8-40, or 8-30 nucleosides long and, in some embodiments, comprise one or more modified nucleosides and/or modified base moieties and/or modified internucleoside linkages. In some embodiments, an antisense oligonucleotide mediates RNaseH activity, which causes degradation of the target mRNA. Antisense oligonucleotides are reviewed, for example, in Antisense Drug Technology, Ed. Stanley T. Corrke, CRC Press, 2007.

In some embodiments, a CMTR1 inhibitor is an siRNA. siRNAs are double-stranded oligonucleotides in which one strand has a nucleobase sequence that permits hybridization to a corresponding segment of a target nucleic acid. siRNAs may comprise various modifications. Such modifications, and siRNAs generally, are well known in the art. See, e.g., siRNA Design: Methods and Protocols, Ed. Debra J. Taxman, Springer-Verlag New York, LLC, 2013.

In some embodiments, a CMTR1 inhibitor is an antibody that binds CMTR1 and inhibits one or more activities of CMTR1. As noted herein, the term “antibody” includes various antibody structures, including but not limited to monoclonal antibodies, polyclonal antibodies, multispecific antibodies (e.g., bispecific antibodies), and antibody fragments so long as they exhibit the desired antigen-binding activity. In some embodiments, a CMTR1 inhibitor is an abzyme.

### E. Exemplary Pharmaceutical Compositions and Routes of Administration

In some embodiments, compositions comprising one or more of the therapeutic agents provided herein are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, pharmaceutical compositions are administered in an amount effective for treatment of (including prophylaxis of) an influenza A infection. The therapeutically effective amount is typically dependent on the weight of the subject being treated, his or her physical or health condition, the extensiveness of the condition to be treated, or the age of the subject being treated.

A therapeutic agent provided herein may be administered in vivo by various routes, including, but not limited to, intravenous, intra-arterial, parenteral, intraperitoneal or subcutaneous. The appropriate formulation and route of administration may be selected according to the particular therapeutic agent and intended application.

### F. Exemplary Combination Therapy

In some embodiments, one or more of the therapeutic agents provided herein is administered in combination with another therapeutic agent. In some embodiments, one or more of the therapeutic agents provided herein is administered in combination with another therapeutic agent for treating influenza A. Nonlimiting exemplary therapeutic agents for treating influenza A that may be administered with the therapeutic agents provided herein include baloxavir marboxil (Xofluza®), oseltamivir (Tamiflu®), and zanamivir (Relenza®). In various embodiments, a therapeutic agent provided herein is administered before, concurrently, or after, administration of another therapeutic agent. In some embodiments, a CMTR1 inhibitor is administered in combination with baloxavir marboxil (Xofluza®).

### G. Embodiments

The following numbered items provide embodiments as described herein, though the embodiments recited here are not limiting.

Item 1. A method of treating influenza A virus infection comprising administering to a subject in need thereof an effective amount of at least one agent selected from a WDR7 inhibitor, a CCDC115 inhibitor, a TMEM199 inhibitor, and a CMTR1 inhibitor.

Item 2. The method of item 1, wherein at least one agent is a WDR7 inhibitor.

Item 3. The method of item 2, wherein the WDR7 inhibitor is selected from an antibody, an antisense oligonucleotide, an siRNA, a peptide, an abzyme, or a small molecule.

Item 4. The method of item 3, wherein the WDR7 inhibitor is a small molecule.

Item 5. The method of item 3, wherein the WDR7 inhibitor is an antisense oligonucleotide or an siRNA.

Item 6. The method of item 5, wherein the antisense oligonucleotide is complementary to a portion of the WDR7 mRNA.

Item 7. The method of item 3, wherein the WDR7 inhibitor is a peptide.

Item 8. The method of item 3, wherein the WDR7 inhibitor is an antibody.

Item 9. The method of item 8, wherein the antibody is an antibody fragment.

Item 10. The method of item 9, wherein the antibody fragment is selected from an scFv, Fab, Fab′, F(ab′)2 fragment.

Item 11. The method of any one of the preceding items, wherein at least one agent is a CCDC115 inhibitor.

Item 12. The method of item 11, wherein the CCDC115 inhibitor is selected from an antibody, an antisense oligonucleotide, an siRNA, a peptide, an abzyme, or a small molecule.

Item 13. The method of item 11, wherein the CCDC115 inhibitor is a small molecule.

Item 14. The method of item 11, wherein the CCDC115 inhibitor is an antisense oligonucleotide or an siRNA.

Item 15. The method of item 14, wherein the antisense oligonucleotide is complementary to a portion of the CCDC115 mRNA.

Item 16. The method of item 11, wherein the CCDC115 inhibitor is a peptide.

Item 17. The method of item 11, wherein the CCDC115 inhibitor is an antibody.

Item 18. The method of item 17, wherein the antibody is an antibody fragment.

Item 19. The method of item 18, wherein the antibody fragment is selected from an scFv, Fab, Fab′, F(ab′)2 fragment.

Item 20. The method of any one of the preceding items, wherein at least one agent is a TMEM199 inhibitor.

Item 21. The method of item 20, wherein the TMEM199 inhibitor is selected from an antibody, an antisense oligonucleotide, an siRNA, a peptide, an abzyme, or a small molecule.

Item 22. The method of item 20, wherein the TMEM199 inhibitor is a small molecule.

Item 23. The method of item 20, wherein the TMEM199 inhibitor is an antisense oligonucleotide or an siRNA.

Item 24. The method of item 23, wherein the antisense oligonucleotide is complementary to a portion of the TMEM199 mRNA.

Item 25. The method of item 20, wherein the TMEM199 inhibitor is a peptide.

Item 26. The method of item 20, wherein the TMEM199 inhibitor is an antibody.

Item 27. The method of item 26, wherein the antibody is an antibody fragment.

Item 28. The method of item 27, wherein the antibody fragment is selected from an scFv, Fab, Fab′, F(ab′)2 fragment.

Item 29. The method of any one of the preceding items, wherein at least one agent is a CMTR1 inhibitor.

Item 30. The method of item 29, wherein the CMTR1 inhibitor is selected from an antibody, an antisense oligonucleotide, an siRNA, a peptide, an abzyme, or a small molecule.

Item 31. The method of item 30, wherein the CMTR1 inhibitor is a small molecule.

Item 32. The method of item 30, wherein the CMTR1 inhibitor is an antisense oligonucleotide or an siRNA.

Item 33. The method of item 32, wherein the antisense oligonucleotide is complementary to a portion of the CMTR1 mRNA.

Item 34. The method of item 30, wherein the CMTR1 inhibitor is a peptide.

Item 35. The method of item 30, wherein the CMTR1 inhibitor is an antibody.

Item 36. The method of item 35, wherein the antibody is an antibody fragment.

Item 37. The method of item 36, wherein the antibody fragment is selected from an scFv, Fab, Fab′, F(ab′)2 fragment.

Item 38. The method of any one of the preceding items, wherein the agent reduces nuclear entry of influenza A.

Item 39. The method of any one of the preceding items, wherein the agent reduces viral replication.

Item 40. The method of item 38 or item 39, wherein the agent is selected from a WDR7 inhibitor, a CCDC115 inhibitor, and a TMEM199 inhibitor.

Item 41. The method of any one of items 1 to 37, wherein the agent reduces viral transcription.

Item 42. The method of item 41, wherein the agent is a CMTR1 inhibitor.

Item 43. The method of any one of the preceding items, wherein the influenza A is selected from H1N1, H3N2, H5N1, and H7N9 influenza A.

Item 44. The method of any one of the preceding items, wherein the influenza A is selected from H1N1 and H3N2 influenza A.

Item 45. The method of any one of the preceding items, wherein the subject is suspected of having an influenza A virus infection.

Item 46. The method of any one of the preceding items, wherein the subject is at risk of developing an influenza A virus infection.

Item 47. The method of any one of the preceding items, wherein the subject has been diagnosed with an influenza A virus infection.

Item 48. The method of any one of the preceding items, wherein the subject exhibits at least one symptom of influenza A virus infection.

Item 49. The method of item 48, wherein at least one symptom is selected from fever, muscle ache, chills, headache, cough, fatigue, nasal congestion, and sore throat.

Item 50. The method of any one of the preceding items, wherein treating influenza A virus infection comprises reducing the severity and/or duration of one or more symptoms of influenza A virus infection.

Item 51. The method of any one of the preceding items, further comprising administering a therapeutic agent selected from baloxavir marboxil, oseltamivir, and zanamivir to the subject.

Item 52. The method of any one of the preceding items, wherein the method comprises administering a CMTR1 inhibitor and baloxavir marboxil to the subject.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Methods

Cell culture and virus strains. A549, A549-cas9 and 293T cells were cultured in Dulbecco's Modified Eagle Medium (DMEM, Thermofisher) supplemented with 10% heat-inactivated fetal bovine serum (Sigma), 2 mM L-Glutamine (Gibco) and 1% penicillin. A549-cas9 cell line was generated by transducing A549 cells with a lentiviral construct (pXPR101) expressing Cas9 and Blasticidin deaminase. Cas9 activity was confirmed by transducing A549-Cas9 cells with a lentiviral construct (pXPR_011-sgEGFP) expressing eGFP and an sgRNA specific for eGFP. Primary Human Lung Fibroblasts (HLF) cells were cultured in Mesenchymal Stem Cell Growth Medium (MSCGM, Lonza). H1N1 (PR8/A/34) and H3N2 (A/Udorn/72) Influenza A viruses were grown in MDCK cells in serum-free DMEM supplemented with 1% BSA and 1 ug/ml TPCK trypsin.

Plasmids. pXPR101 and pXPR_011-sgEGFP used to generate A549-Cas9 cells and pLentiGuide-puro (Addgene #52963) for secondary screen were provided by the Broad Institute Genetic Perturbation Platform. Individual sgRNAs were cloned into pLentiCRISPR-V2 (Addgene #52961) and pXPR_004 (Puromycin resistance gene in pLentiCRISPR-V2 was replaced by eGFP) for validation in A549 cells and HLFs respectively. For rescue experiments, Cas9 gene in pXPR101 was replaced by codon-mutated versions of WDR7, CCDC115, TMEM199 and CMTR1 genes (pXPR101_rescue). For pseudovirus production, MLV Gag-pol, GFP, PR8 HA, PR8 NA and MLV Env plasmids were kindly provided by Marci DeGrace.

Antibodies. The following antibodies were used: From EMD Millipore, Anti-Influenza A HA (AB1074), FITC Anti-Influenza A Nucleoprotein clone A1 (MAB8257F). From Abcam, Anti-LAMP1 clone H4A3 (ab25630), Anti-Rab7 Alexa-Fluor647 clone EPR7589 (ab198337), β-actin antibody (ab6276). From BD bioscience, FITC mouse anti-human CD71 (555536). From Thermofisher, Alexa-Fluor488 Goat anti-mouse IgG, Alexa-Fluor488 Donkey anti-goat IgG. From Sigma Aldrich, Anti-Flag M2 antibody (F3165).

Pooled genome-wide CRISPR screen. 100 million A549-Cas9 cells were transduced with the AVANA-4 lentiviral library (Doench J G, et al. Nat Biotechnol. 2016; 34(2): 184-191) to achieve 40% infection rate and average 500-fold coverage of the library after selection. After 24 hours, the cells were selected with puromycin and an initial pool of 40 million cells were harvested for genomic DNA extraction using the Qiagen Blood and Tissue extraction kit according to manufacturer protocol. On day 9 post-transduction, 200-400 million puromycin resistant A459-Cas9 cells were infected with Influenza A PR8 virus at MOI5 for 16 hours. Cells were then washed and stained with anti-Influenza A HA (AB1074) antibody. HA-positive and HA-negative cells were sorted by FACS and harvested for genomic DNA. PCR and sequencing were performed as previously described (Doench et al, 2016).

sgRNA library cloning and lentiviral production. The AVANA-4 library (74,700 sgRNAs targeting 18,675 genes and 1000 non-targeting sgRNA) was provided by the Broad Institute Genetic Perturbation Platform. For the secondary screen, a plasmid library containing 18,870 sgRNAs targeting the top 1000 ranked genes from the primary screen and 787 genes from cross-validation analysis as well as 1000 non-targeting sgRNAs was synthesized as oligonucleotides (Broad Institute Biotechnology Lab). The sgRNAs were cloned by Gibson Assembly into the pLentiGuide-Puro vector and lentivirus was produced from 293T cells as previously described (Doench et al, 2016).

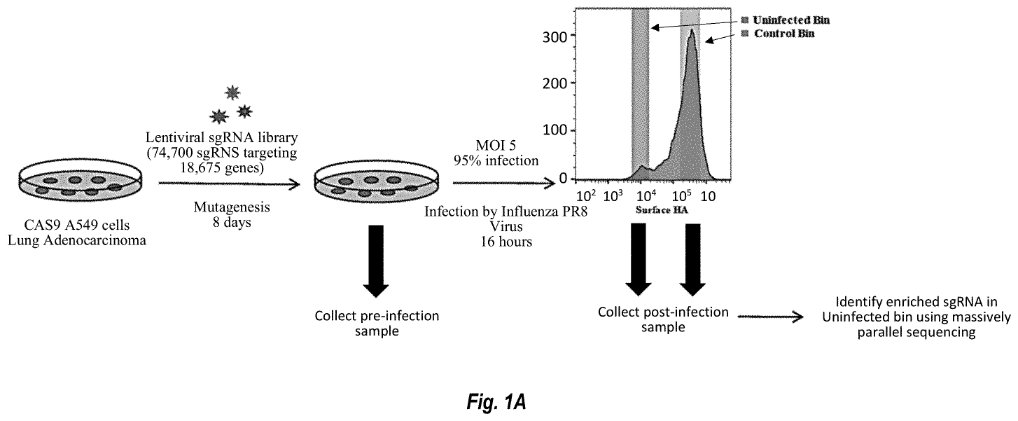

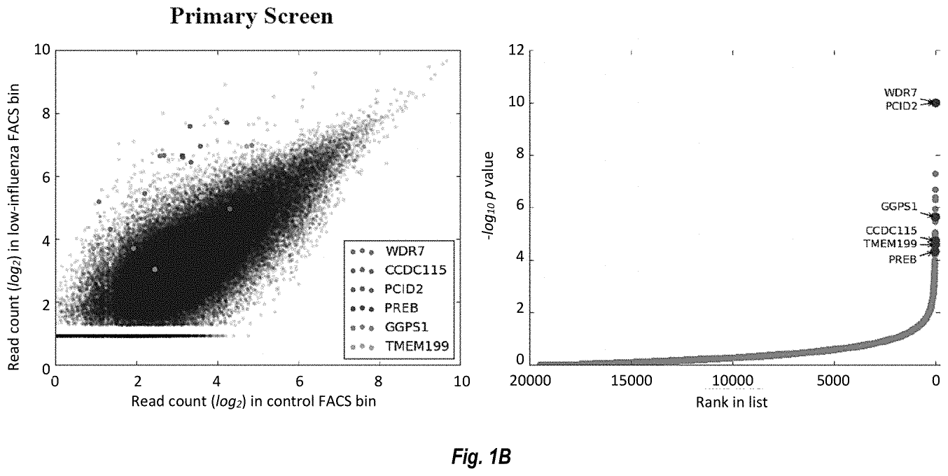

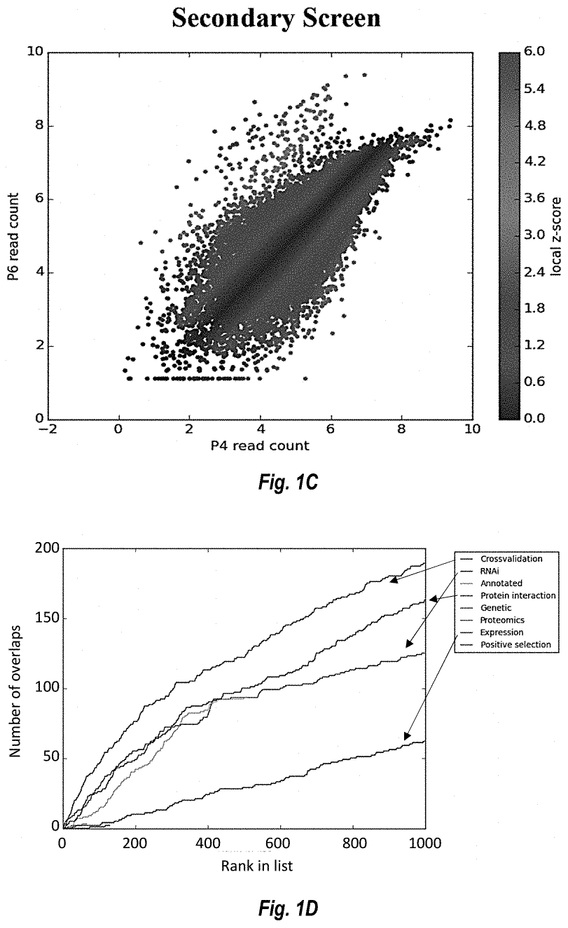

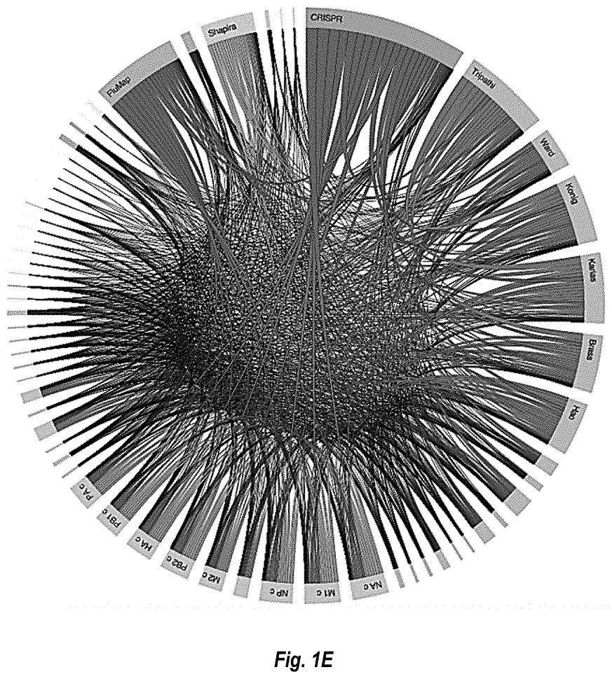

Screen analysis. Read counts corresponding to each guide RNA were normalised to reads per million and log transformed. Quantile normalisation was performed in R. In order to control for the marked heteroscedasticity (FIG. 1B), local z-scores, for pools of values with different read counts, were calculated for sliding bins of varying size. For any comparison of two samples from which n read counts [x] and [y] are derived (for example, the flu-permissive and control FACS pools), the null hypothesis is xᵢ=yᵢ, where i is the ranked position in the list of read counts. The read count bin was determined from the shortest distance between any point (xᵢ,yᵢ) and the line y=x. Lower (l) and upper limits of n sliding bins of size b were defined such that each bin contains b values. Z-scores were then calculated within each of these bins. These z-scores were converted to empirical p-values and combined (Σ−log₁₀(p); Fisher's method). In order minimize false negatives and maximize the discovery power of our screen, we did not require more than one sgRNA per gene to be significantly over-represented in the influenza virus-permissive FACS pool.

Cross-validation analysis. In a set of input lists of named entities (in the present work, genes, but any entity with a unique name could be used here), Integrative cross-validation aims first to evaluate the information content in each list by comparing it to all other lists, and then summarize the evidence in support of each entity from all lists taken together. This is achieved using an unbiased approach, agnostic to the source of data in each input set. The weighting given to each data set is determined by how well-supported are the constituent members of that set. Each entity (or gene) is given a score that reflects the quality and quantity of evidence in support of it: the sum of the weighting scores for each of the lists in which that gene is found. The end result reflects the composite evidence from all of the input sources in support of a given entity.

Validation of individual hits using gene-specific CRISPR sgRNA. For validation of individual hits in A549 cells, the two best sgRNAs from the AVANA-4 library were cloned into pLentiCRISPR-V2 and lentivirus was produced from 239T cells as previously described (Doench et al, 2016). A549 Cells were transduced and selected with 1 ug/ul puromycin for 8 days and genome-editing was confirmed by deep sequencing. For validation in HLF cells, sgRNAs were cloned into pXPR_004, which carries eGFP instead of a puromycin resistance gene. Following transduction, GFP+ HLF cells were sorted by FACS. Transduced cells were infected with influenza A virus for 16 hours at MOI5 and stained for surface HA using anti-influenza A HA antibody (AB1074).

Rescue and over-expression of knock-out (KO) genes. A549 cells were transduced with pLentiCRISPR-V2 expressing a gene-specific sgRNA together with a XPR101_rescue plasmid expressing Flag-tagged codon-mutated version of the gene. Cells were selected with 1 ug/ul puromycin and 10 ug/ul blasticidin for 8 days. Expression of the add-back gene was confirmed by Western blot. To test if the genes of interest have redundant functions, A549 cells were transduced with different combinations of the gene-specific sgRNAs and codon-mutated versions of the genes. To test the effect of over-expressing the genes alone, A549 cells were transduced with the rescue plasmids in the absence of sgRNAs.

Influenza A virus infection. A549 or HLF cells were seeded on 12 well plates and inoculated with Influenza A PR8 (H1N1) or Udorn virus (H3N2) at MOI 5 for 1 hour at 37° C. in serum-free DMEM. The cells were then washed and replaced with fresh serum-free DMEM supplemented with 1% BSA for 16 hours. Infection was subsequently monitored by FACS or plaque assay.

Flow cytometry. Cells were stained with antibodies in PBS+1% BSA for 30 minutes on ice and fixed with 4% paraformaldehyde. For intracellular staining for Influenza A nucleoprotein (NP), cells were fixed and permeabilized using 0.1% saponin (Sigma Aldrich) prior to antibody staining. Data were acquired on the BD Accuri (Bd Bioscience) and analyzed by FlowJo software (TreeStar).

Plaque assays. A549 or HLFs cells were infected with Influenza A PR8 (H1N1) or Udorn virus (H3N2) at MOI 0.1 in serum-free DMEM with 1% BSA and 1 ug/ul TPCK trypsin. 48 hours post-infection, supernatant was collected and serial-diluted. 200 ul of the diluted supernatant was used to infect MDCK cells on 6 well plates and the number of plaques were counted after 72 hours. The virus titer was calculated in plaque forming units (PFU)/ml.

Proliferation assays. A549 cells were transduced with pLentiCRISPR-V2 expressing sgRNA against genes of interest and selected with 1 ug/ul puromycin for 2 days. On day 3, 5000 puromycin resistant cells were re-seeded on 6-well plates and change in cell number were monitored on day 5, 7 and 9. On day 9, some cells were also harvested for ALAMAR Blue assay (Thermofisher, DAL1025) and Annexin V staining (Thermofisher, V13241) according to manufacturer protocol.

MLV-GFP pseudovirus production and entry assay. MLV-GFP pseudovirus was produced as previously described (Huang et al, J Virol. 2008; 82(10):4834-43). Briefly, Gag-pol and GFP expressing plasmids were transfected into 293T cells together with PR8 HA and NA or MLV-Env. Virus was harvested and filtered 48 hours post-transfection. To test for entry, A549 cells were spinoculated with the pseudovirus at 2000 rpm for 30 minutes. GFP expression was monitored 48 hours post-spinoculation by FACS.

Influenza A virus binding assay. Cells were seeded on 6 well-plates and inoculated with Influenza A PR8 virus at MOI 100 for 30 minutes at 4° C. They were then washed twice with ice cold PBS and stained for surface HA using anti-influenza A HA antibody (AB1074).

Measuring level of cell surface sialic acid. Cells were stained with Sambucus nigra lectin (SNA) (Vector Laboratories Inc.) according to the manufacturer protocol. Briefly, Cells were incubated with 10 ug/ml FITC-conjugated Lectin at room temperature for 30 minutes. They were then washed twice in PBS and analyzed by FACS.

Fluorescent in situ hybridization (FISH). 1×10\hat{ }5 cells were seeded on a chambered cover glass (VWR, Nunc Lab-Tec 2 wells) pre-treated with 0.1 mg/ml poly-D-lysine. The cells were infected with Influenza A PR8 virus the following day for 4 hours at 37° C. Cells were then fixed and stained with Stellaris Quasar 570 RNA FISH probes against Influenza A PR8 NP RNA according to manufacturer protocol (LGC Biosearch Technologies). Images were taken on the Olympus FV1200 IX83 confocal microscope and percentage of RNA+ cells relative to the total number of cells was quantified.

Confocal microscopy. Cells were imaged on the Olympus FV1200 IX83 laser scanning confocal microscope equipped with a 40× objective and LD559, LD635 and LD405 (Olympus Life Science). Images were taken using the Olympus FV software and analyzed using ImageJ.

For imaging of LysoTracker/LysoSensor, Rab7 and LAMP1, 5×10\hat{ }4 A549/HLF cells were seeded onto chambered cover glass (VWR, Nunc Lab-Tec 4 wells) pre-treated with 0.1 mg/ml poly-D-lysine the day before. Cells were treated with 100 nM LysoTracker dye for 1 hours at 37° C., followed by fixation with 4% paraformaldehyde and permeabilization with 0.1% saponin. The cells were blocked with PBS with 1% BSA and 0.1% Tween-20 for 1 hour at room temperature and stained with anti-Rab7 and anti-LAMP1 antibodies overnight at 4° C. The cells were then stained with secondary Alexa-fluor488-conjugated goat anti-mouse IgG antibody and DAPI for 1 hour at room temperature. Images were acquired with a 40× objective using the setup described above.

For visualization of Influenza NP localization within the cells, A549 cells were infected with Influenza A PR8 virus at MOI 200 for 2 hours at 37° C. Infected cells were then fixed with 4% paraformaldehyde and stained with FITC anti-influenza A NP antibody overnight at 4° C. The next day cells were washed, stained with DAPI for 1 hour at room temperature and images were acquired as described above.

Transferrin uptake assay. To test for endocytosis of transferrin, A549 cells were incubated with Alex-Fluor647-conjugated transferrin (Thermo Fisher, T23366) for 30 minutes at 37° C. The cells were then washed, fixed in 4% paraformaldehyde and stained with FITC anti-transferrin receptor (CD71) antibody overnight at 4° C. The next day cells were washed and stained with DAPI for 1 hour at room temperature. Images were acquired as described before and the ratio of internalized transferrin versus membrane transferrin was quantified.

Measuring degradation of Influenza NP RNA. To measure the rate of degradation of Influenza RNA upon infection, A549 cells were seeded on 12-well plates and incubated with Influenza A PR8 virus at MOI 100. Cells were lysed and RNA was extracted at 10, 30, 60 and 90 minutes post-infection using the RNeasy Mini Kit (Qiagen). To prevent de novo synthesis of viral RNA, cells were treated with 40 uM importazole to block viral entry into the nucleus. cDNA synthesis and qRT-PCR for PR8 NP was performed as described herein.

Measuring lysosomal degradation of DQ-BSA. 1×10\hat{ }5 A549 cells were seeded on 12-well plates and incubated with 20 ug/ml DQ Green BSA (Thermo Fisher, D12050), 100 nM LysoTracker Red and DAPI for 1 hour at 37° C. The cells were then washed in PBS and fixed in 4% paraformaldehyde. Confocal microscopy Images were acquired with a 40× objective using the setup described earlier.

Luciferase reporter assay for Influenza A virus replication. To measure viral polymerase activity, a vRNA-luciferase reporter system previously developed in the lab was utilized (Shapira et al, Cell. 2009; 139(7):1255-67). Briefly, A549 cells were transfected with a vRNA reporter plasmid expressing firefly luciferase under a viral UTR. The cells were also transfected with Influenza A virus PA, PB1, PB2, NP and Renilla. 24 hours post-transfection, cells were lysed and mixed with Dual Glo substrate (Promega) according to Manufacturer's protocol. Luminescence was measured and quantified using a Synergy H1 multi-mode microplate reader (BioTek).

RNA extraction and qPCR. Total RNA was extracted from 1×10\hat{ }5 cells using the RNeasy Mini Kit (Qiagen) according to manufacturer's protocol. First strand cDNA synthesis was performed using 500 ng of total RNA with the Superscript III First-strand Synthesis system with Oligo(dT) (Thermo Fisher). Quantitative qPCR was performed using the Q5 hot start high fidelity polymerase and SYBR green I Nucleic Acid Gel stain (Thermo Fisher) on the Roche 480 Light Cycler (Roche). Human GAPDH was used as reference normalization control and expression levels were quantified by the delta Ct method. Primer sequences are as follows:

Human IFN-β

Forward: 5′-TGCTCTCCTGTTGTGCTTCT-3′ (SEQ ID NO: 1)

Reverse: 5′-ATAGATGGTCAATGCGGCGT-3′ (SEQ ID NO: 2)

Influenza PR8 NP

Forward: 5′-ATCGGAACTTCTGGAGGGGT-3′ (SEQ ID NO: 3)

Reverse: 5′-CAGGACTTGTGAGCAACCGA-3′ (SEQ ID NO: 4)

Human GAPDH

Forward: 5′-GGGAGCCAAAAGGGTCATCA-3′ (SEQ ID NO: 5)

Reverse: 5′-AGTGATGGCATGGACTGTGG-3′ (SEQ ID NO: 6)

RNA sequencing. Transcriptomic analysis was performed using the Smart-Seq2 protocol described previously. Briefly, total RNA was extracted using the RNeasy Mini Kit (Qiagen). cDNA was synthesized from 1 ng of total RNA using the SuperScript III reverse transcription system, followed by PCR pre-amplification and quality check using high-sensitivity DNA Bioanalyzer chip (Agilent). 0.15 ng of pre-amplified cDNA was then used for the tagmentation reaction carried out with the Nextera XT DNA sample preparation kit (Illumina) and final PCR amplification. Amplified library was sequenced on a HiSeq 2500 (Illumina). For data analysis, short sequencing reads were aligned using Bowtie2 and used as input in RSEM to quantify gene expression levels for all UCSC hg19 genes. Data were normalized and analyzed using the R software package DESeq2.

Western blotting. To check for expression of WDR7, CCDC115, TMEM199 and CMTR1 expression in rescue experiments, 5×10\hat{ }5 transduced cells were washed with ice-cold PBS and lysed in RIPA buffer (Thermo Fisher)+1 tablet of EDTA-free protease inhibitor cocktail (Roche) per 25 ml buffer. Cell lysates were span at 12,000 rpm in a microcentrifuge for 10 minutes at 4° C. and denatured by heating at 95° C. in SDS loading buffer+DTT. Proteins were separated on a NuPAGE Novex 12% Tris-Glycine gel and transferred to a polyvinylidene difluoride membrane (Milipore). Immunoblotting was performed according to standard protocols using rabbit anti-Flag primary antibody and HRP-conjugated anti-rabbit secondary antibody.

### Example 2: Genome-Wide CRISPR Screens Identify Novel Influenza a Virus (IAV) Host Dependency Factors

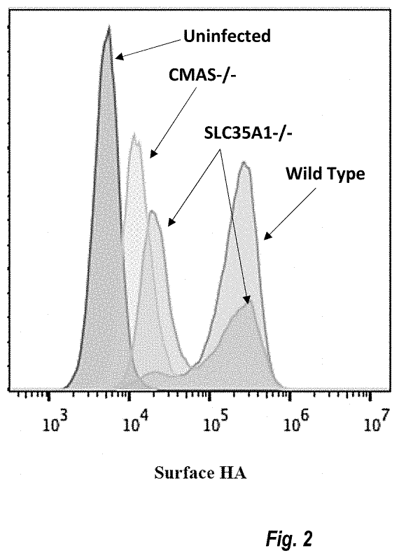

Two independent rounds of pooled genome-wide screens in A549-Cas9 cells were performed using the AVANA4 lentivirus library, which expresses 74,700 sgRNAs targeting 18,675 annotated protein-coding genes (4 sgRNAs per gene), as well as 1,000 non-targeting sgRNAs as control. (Doench J G, et al. Nat Biotechnol. 2016; 34(2): 184-191.) On day 9 post-transduction with the library, ˜300 million puromycin-resistant cells were infected with influenza PR8 virus at MOI 5 for 16 hours. Cells were sorted into 3 bins based on their surface hemagglutinin (HA) expression (FIG. 1A, FIG. 2). Approximately 5% of the cells were sorted into the uninfected bin, which were more likely to contain genetic perturbations that inhibit IAV infection. sgRNAs in each bin were sequenced and the relative abundance analyzed using a modified version of the STARS algorithm. (Doench et al. 2016.)

sgRNAs targeting 41 genes were identified as significantly enriched in the uninfected bin relative to control bin (FDR<0.05) ((FIG. 1B). These included known host dependency factors from previous RNAi screens, including V-type ATPase subunits (rank 1, 2, 3, 4, 8, 9, 12, 13, 19, 21, 23), components of the vesicular transport pathway (rank 14, 27, 41), signal recognition particles (rank 17, 23), and genes involved in sialic acid synthesis (rank 7, 21) (Table 1). Interestingly, numerous hits that have not been discovered in previous siRNA screens were also identified. These included components of the TRAPP complex (rank 33, 39, 40) and TREX2 complex (rank 6), genes involved in protein prenylation (rank 10, 19) and recently described co-factors (rank 5, 26, 29) of the V-type ATPases. Components of the TREX2 complex (PCID2, MCM3AP, SHFM1), which interacts with NXF1 to transport nascent messenger ribonucleoprotein particle (mRNP) from the nucleus to the cytoplasm. While NXF1 has been identified in this and numerous RNAi screens as an IAV dependency factor, prolonged deletion of NXF1 was found to be lethal in A549 cells. Interestingly, the TREX2 complex only transport a subset of the transcripts that are being transported by NXF1 and results in a milder phenotype when knocked down by siRNA. This suggests that components of the TREX2 complex may serve as better therapeutic targets than NXF1. In addition, numerous enzymes involved in the protein prenylation pathway were identified, including the geranylgeranyl Diphosphate synthase 1 (GGSP1) and Rab Geranylgeranyltransferases (RABGGTase). GGPS1 is required for the synthesis of geranylgeranyl diphosphates while RABGGTase are needed to transfer the geranylgeranyl groups to Rab proteins. Both enzymes are required for proper homing of Rab proteins to their respective intracellular compartments.

TABLE 1
Genes enriched in uninfected bin relative to control bin

| gene | p-value | fdr |
|---|---|---|
| ATP6V1F | <10E−12 | <10E−12 |
| ATP6V1B2 | <10E−12 | <10E−12 |
| ATP6V0B | <10E−12 | <10E−12 |
| ATP6V0C | <10E−12 | <10E−12 |
| WDR7 | <10E−12 | <10E−12 |
| PCID2 | <10E−12 | <10E−12 |
| SLC35A1 | <10E−12 | <10E−12 |
| ATP6AP2 | <10E−12 | <10E−12 |
| ATP6AP1 | <10E−12 | <10E−12 |
| RABGGTB | <10E−12 | <10E−12 |
| ATP6V1A | <10E−12 | <10E−12 |
| ATP6V1E1 | <10E−12 | <10E−12 |
| COG3 | <10E−12 | <10E−12 |
| DBR1 | <10E−12 | <10E−12 |
| SRP14 | <10E−12 | <10E−12 |
| ATP6V1H | <10E−12 | <10E−12 |
| ATP6V1G1 | <10E−12 | <10E−12 |
| ATP6V1C1 | <10E−12 | <10E−12 |
| CCDC115 | <10E−12 | <10E−12 |
| ATP6V1D | <10E−12 | <10E−12 |
| CMAS | <10E−12 | <10E−12 |
| GGPS1 | 8.85966E−12 | 7.8344E−09 |
| DPAGT1 | 5.17773E−11 | 4.3795E−08 |
| TMEM199 | 2.20916E−10 | 1.7907E−07 |
| COG4 | 7.51403E−10 | 5.8471E−07 |
| PREB | 1.0921E−09 | 8.1714E−07 |
| SRPRB | 2.66756E−09 | 1.922E−06 |
| SRP54 | 6.33316E−09 | 4.4002E−06 |
| YKT6 | 1.95E−08 | 1.3081E−05 |
| NSF | 2.06433E−08 | 1.3387E−05 |
| COG8 | 2.30535E−08 | 1.4467E−05 |
| TRAPPC4 | 2.51681E−08 | 1.5301E−05 |
| COG2 | 2.89917E−08 | 1.7091E−05 |
| TRAPPC5 | 5.93933E−08 | 3.3983E−05 |
| EEF2 | 1.53924E−07 | 8.5555E−05 |
| NUDT21 | 2.22106E−07 | 0.00012002 |
| ATP6V0D1 | 2.54727E−07 | 0.00013393 |
| TRAPPC8 | 3.26807E−07 | 0.00016731 |
| TRAPPC3 | 3.43047E−07 | 0.00017112 |
| RCC1 | 3.94929E−07 | 0.00019207 |
| TPR | 4.06352E−07 | 0.00019281 |
| TNFAIP3 | 4.32929E−07 | 0.00020053 |
| MCM3AP | 4.81334E−07 | 0.00021525 |
| SCAP | 4.86839E−07 | 0.00021525 |
| SRP19 | 6.80421E−07 | 0.00029415 |
| SPCS3 | 7.32777E−07 | 0.0003099 |
| RABGGTA | 1.024E−06 | 0.00042385 |
| ALG2 | 1.25113E−06 | 0.00050707 |
| CMTR1 | 1.7809E−06 | 0.0007067 |
| TRAPPC1 | 1.81634E−06 | 0.0007067 |
| COPG1 | 1.85823E−06 | 0.00070882 |
| COG1 | 1.98135E−06 | 0.00071602 |
| SHFM1 | 1.9875E−06 | 0.00071602 |
| TRAPPC11 | 1.97907E−06 | 0.00071602 |
| COPA | 2.77787E−06 | 0.00098256 |
| EXOC3 | 3.30107E−06 | 0.00114677 |
| SCFD1 | 4.64342E−06 | 0.00158479 |
| SYS1 | 6.14685E−06 | 0.00206174 |
| SRRT | 6.45093E−06 | 0.00211416 |
| SEC16A | 6.52048E−06 | 0.00211416 |
| NUP85 | 8.92209E−06 | 0.00284542 |
| RPN2 | 1.07923E−05 | 0.00338634 |
| SFPQ | 1.12091E−05 | 0.0034613 |
| STX18 | 1.27449E−05 | 0.00387406 |
| RPL31 | 1.32574E−05 | 0.00396784 |
| PAXBP1 | 1.43829E−05 | 0.00421424 |
| RANGAP1 | 1.45139E−05 | 0.00421424 |
| EXOC4 | 1.76789E−05 | 0.00500079 |
| RPL6 | 1.77369E−05 | 0.00500079 |
| POLE2 | 2.40694E−05 | 0.00668923 |
| SAP30BP | 2.49629E−05 | 0.00683982 |
| NXF1 | 3.23461E−05 | 0.00873972 |
| BCL2L1 | 3.39068E−05 | 0.00903594 |
| NCBP2 | 4.09983E−05 | 0.01077811 |
| IPO13 | 5.01877E−05 | 0.01301803 |
| SARS | 5.17259E−05 | 0.01316197 |
| SBDS | 5.20958E−05 | 0.01316197 |
| SRP9 | 6.25132E−05 | 0.01559143 |
| MYH9 | 6.33407E−05 | 0.01559784 |
| TUBGCP2 | 6.50007E−05 | 0.01580653 |
| TMEM38A | 7.0716E−05 | 0.01698407 |
| GOSR2 | 8.18615E−05 | 0.01926837 |
| RARS2 | 8.2208E−05 | 0.01926837 |
| BET1 | 8.58772E−05 | 0.01988875 |
| SOCS3 | 8.99984E−05 | 0.02059799 |
| FAU | 9.23285E−05 | 0.02088557 |
| POLR3H | 9.55719E−05 | 0.02137075 |
| DAD1 | 9.76553E−05 | 0.02158849 |
| TWISTNB | 0.000103518 | 0.02262751 |
| ACTB | 0.000107571 | 0.02316008 |
| TSG101 | 0.000108336 | 0.02316008 |
| NUP43 | 0.00013378 | 0.0282332 |
| RIOK1 | 0.000135895 | 0.0282332 |
| FAM86B1 | 0.00013642 | 0.0282332 |
| BUB3 | 0.000141461 | 0.02874933 |
| RPLP0 | 0.00014187 | 0.02874933 |
| FAM25A | 0.000144483 | 0.02897701 |
| C21orf59 | 0.000146793 | 0.02913997 |
| HAUS3 | 0.000153538 | 0.02986932 |
| RPS27A | 0.000152924 | 0.02986932 |
| INTS6 | 0.000159135 | 0.03065161 |
| CCT4 | 0.000171889 | 0.03248708 |
| TULP1 | 0.000172004 | 0.03248708 |
| CHMP6 | 0.000174262 | 0.03259713 |
| RNGTT | 0.000176143 | 0.03263514 |
| SLC39A7 | 0.000183366 | 0.0334088 |
| ENY2 | 0.000183754 | 0.0334088 |
| KCNG2 | 0.000186158 | 0.03353265 |
| LRPPRC | 0.000206424 | 0.03684201 |
| CENPW | 0.000214399 | 0.03791736 |
| SEC13 | 0.000216461 | 0.03793727 |
| SMARCB1 | 0.000226727 | 0.03895887 |
| INTS4 | 0.000228298 | 0.03895887 |
| PRPF31 | 0.000225251 | 0.03895887 |
| TTC27 | 0.00023426 | 0.03962859 |
| RANBP2 | 0.000243952 | 0.04091248 |
| TUBGCP6 | 0.000254284 | 0.04228065 |
| CSE1L | 0.000257268 | 0.04241427 |
| ANKLE2 | 0.000263344 | 0.04305129 |
| PALM | 0.000275393 | 0.04464574 |
| POLR3A | 0.000283357 | 0.04555729 |
| PKD1 | 0.000285805 | 0.04557418 |
| FOXD4 | 0.000289585 | 0.04580147 |
| EIF3G | 0.000316248 | 0.04882763 |
| ESPL1 | 0.000313682 | 0.04882763 |
| ZNHIT2 | 0.000314391 | 0.04882763 |
| NIPSNAP3B | 0.000318813 | 0.04883609 |
| RNASEK | 0.000322649 | 0.04903758 |

Unlike previous RNAi screens, relatively few ribosomal subunits and genes involved in translation and splicing among were found in the top ranked hits, suggesting that CRISPR is optimized to eliminate host factors essential for cell survival. However, no genes that restrict IAV infection (sgRNAs that would be enriched in the super-infected bin) were found, which could be due to HA expression already being saturated at MOI 5. In addition, CRISPR deletion of PRPF8 and STARD5, two genes which have been identified in least 3 RNAi screen, was found to be lethal in this system. (Chou Y Y, et al. PLoS Pathog. 2013; 9(5):e1003359.)

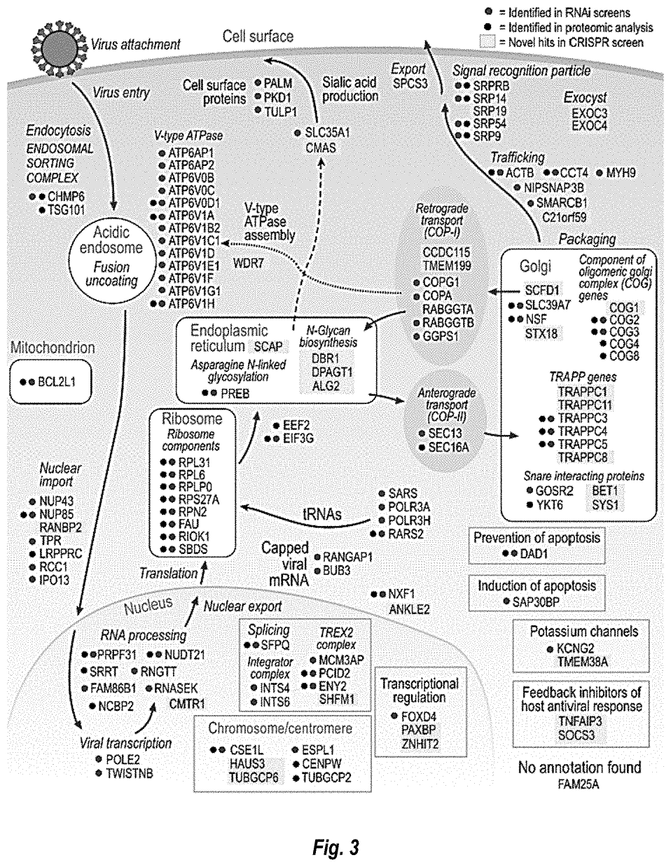

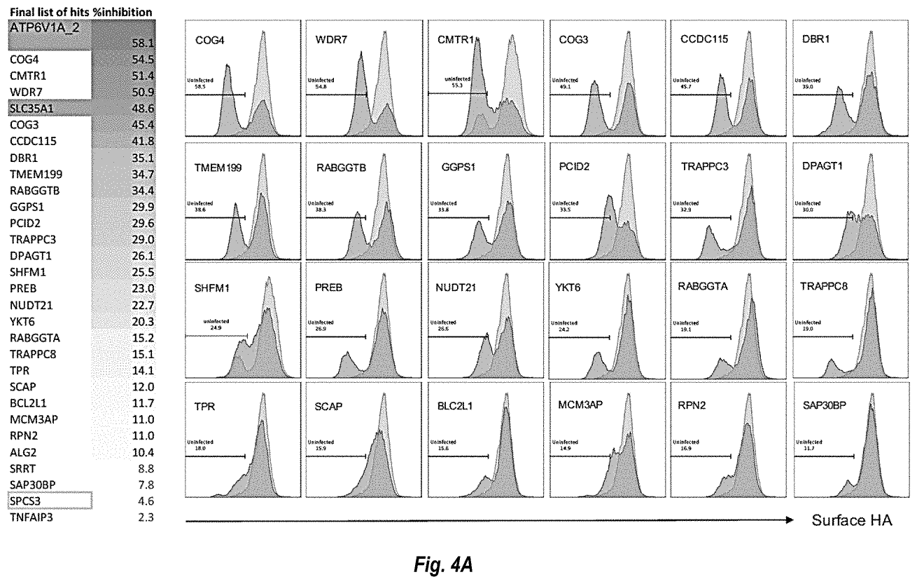

To further confirm the results, a secondary pooled screen targeting the top 1,000 ranked hits from the primary screen but with 10 sgRNAs per gene was performed. Significantly more sgRNAs were found that were enriched in the uninfected bin compared to primary screens (FIG. 1C). 37 out of the 41 hits with FDR<0.05 were re-identified from the primary screen, as well as additional hits that failed to meet the FDR cutoff. Combining data from the primary and secondary screens yielded a final list of 128 hits (FDR<0.05) that are involved in different stages of the IAV life-cycle (FIG. 3). To compare the study with previous siRNA screens, an integrative cross-validation approach was utilized and it was found that the present study provides the most information when compared with previous genetic screens. (FIG. 1D, FIG. 1E).

### Example 3: Validation of Screen Hits in A549 and Primary Human Lung Fibroblast (HLF) Cells

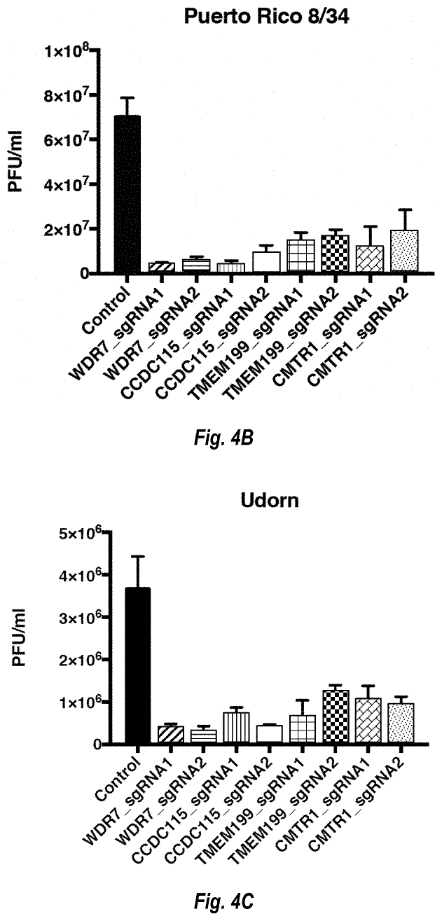

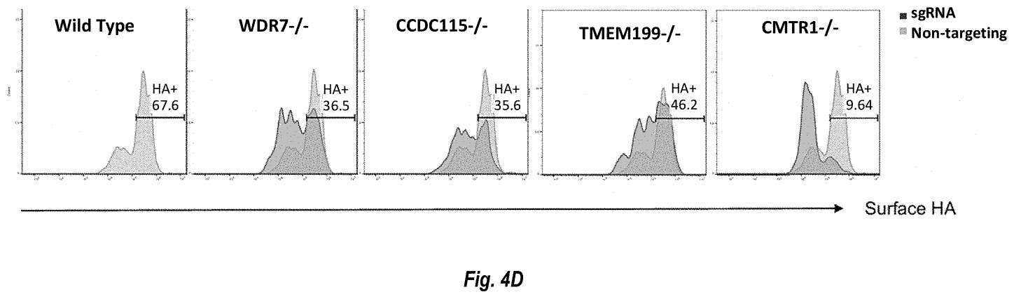

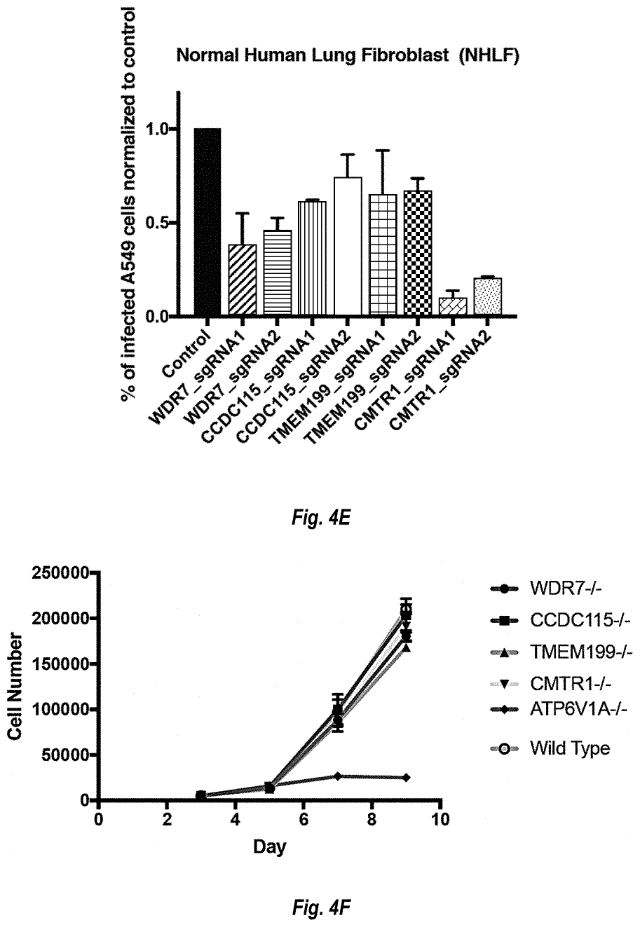

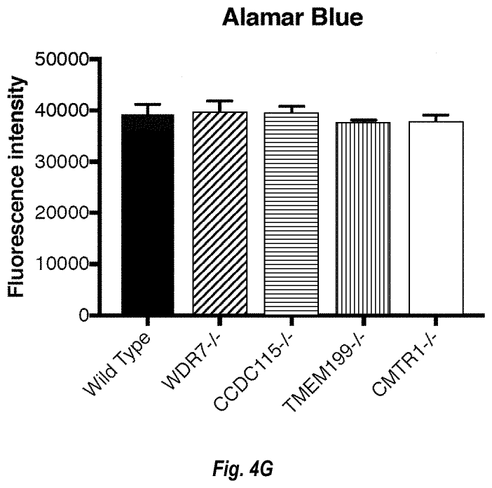

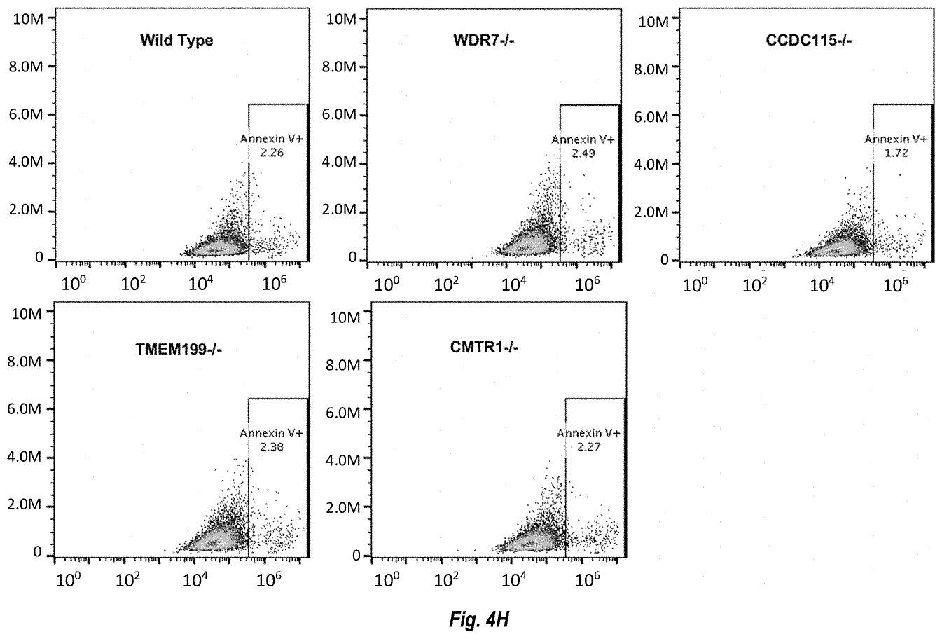

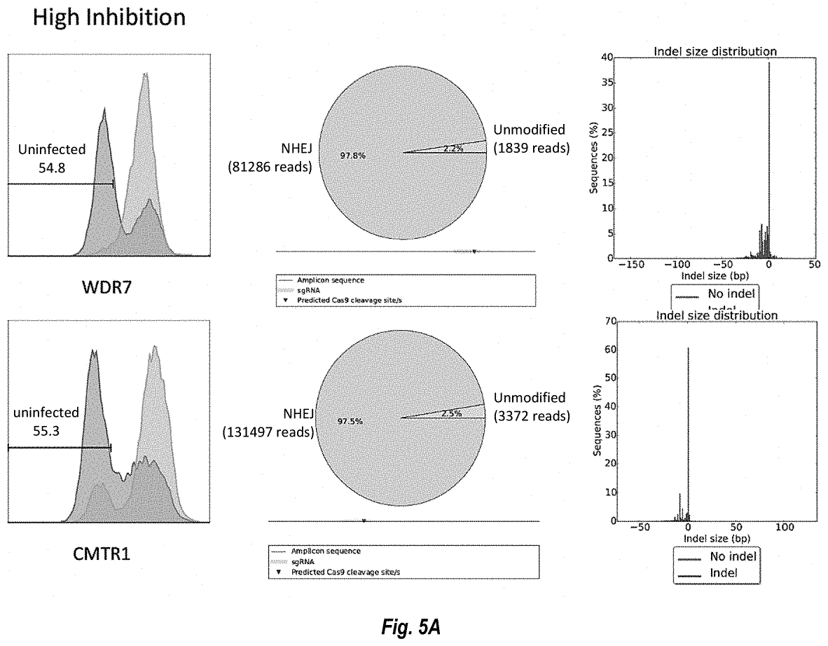

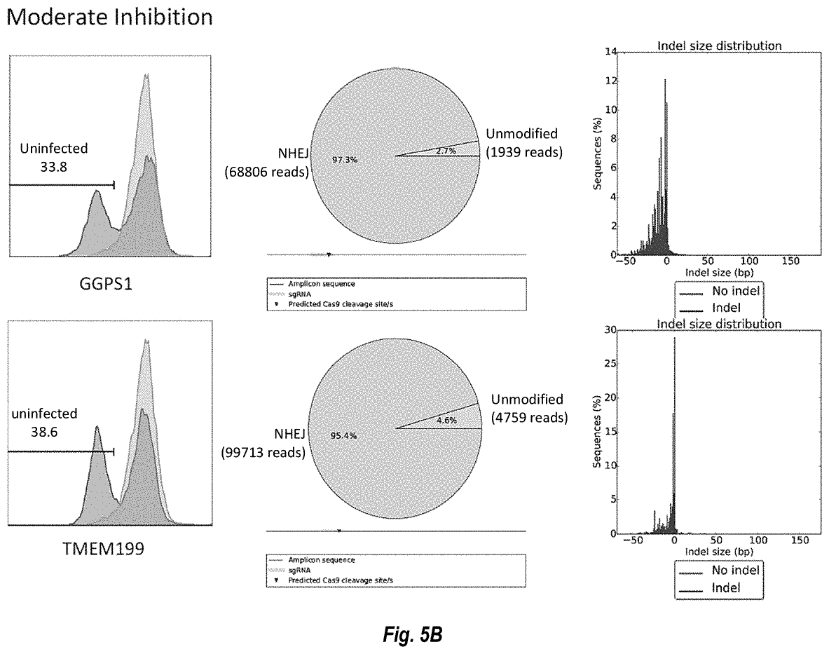

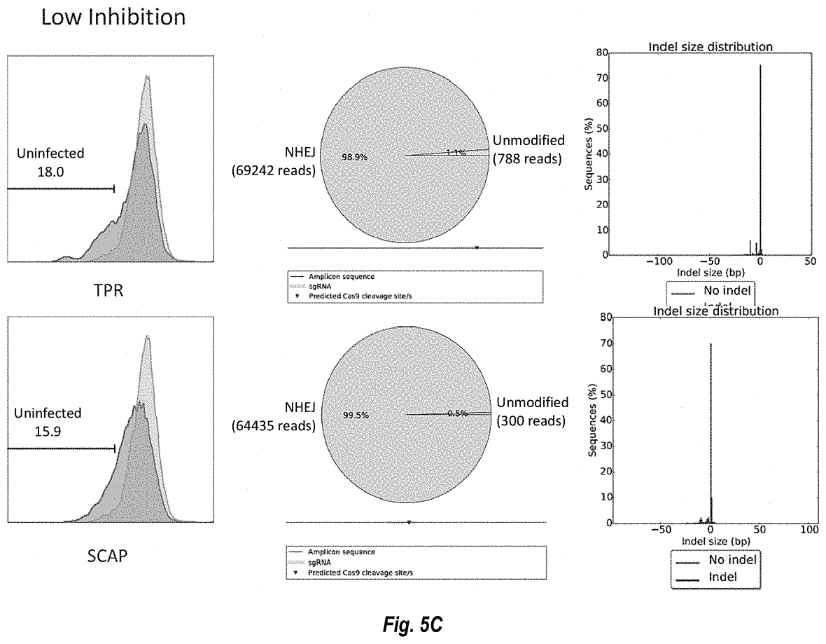

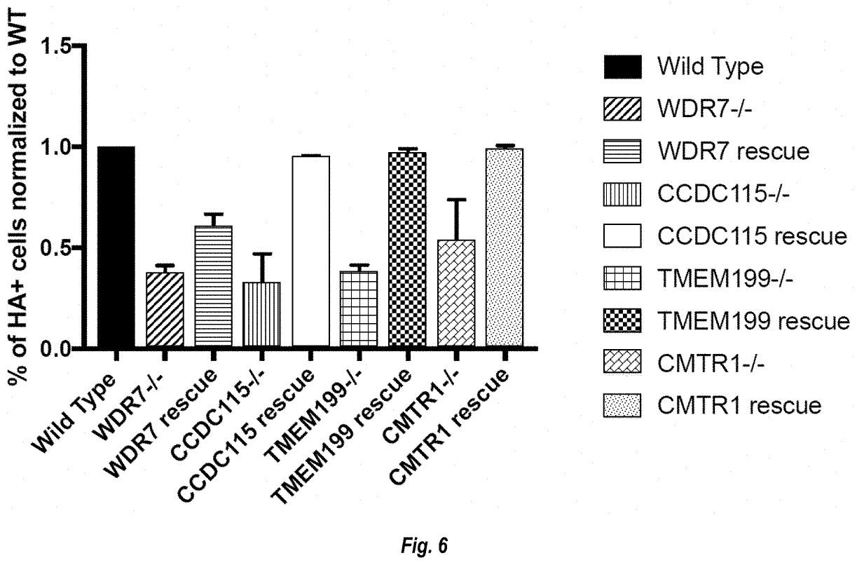

Twenty-eight genes that have not been previously implicated in IAV infection were selected for further validation. Individual genes were knocked out in A549 cells using the top 2 sgRNAs from the secondary screen and genome editing was confirmed by massively parallel sequencing of the predicted target site. Puromycin-resistant cells were then infected with Influenza PR8 virus on day 9 post-sgRNA transduction and stained for surface HA. 24 out of the 28 selected genes protected A549 cells against PR8 virus infection when they were knocked out (FIG. 4A). The degree of protection varied between different hits despite the sgRNAs having comparable genome editing efficiency (FIG. 5), suggesting that the genes differ in their importance for IAV infection. Four of the hits, WDR7, CCDC115, TMEM199 and CMTR1 strongly protected A549 cells against PR8 infection (>30% reduction in percentage of HA+ cells) and had minimum impact on cell proliferation up to 9 days post-sgRNA transduction when they were knocked out (FIG. 4F). Annexin V and ALAMAR blue assay also revealed similar cell states between KO and wild type cells on day 9 post-transduction (FIG. 4G, FIG. 4H). To test if these genes are important for virus production, KO and wild type cells were infected at MOI 0.1 and supernatants were collected at 48 hours post-infection. Supernatants were then used for plaque assays in MDCK cells. All four KO cell lines produced 5-10 fold lower virus titers compared to wild type cells (FIG. 4B). Similar requirement for IAV infection were observed for all four genes in HLFs cells and against H3N2 virus infection, suggesting that their roles extend to other cell types and IAV strains (FIG. 4C, FIG. 4D). Furthermore, re-expression of sgRNA-resistant copies of the genes in the KO cells restored IAV infection level, indicating that the phenotype is not due to off-target effects. (FIG. 6).

### Example 4: WDR7, CCDC115, TMEM199 and CMTR1 are Involved in Early Stages of IAV Life Cycle

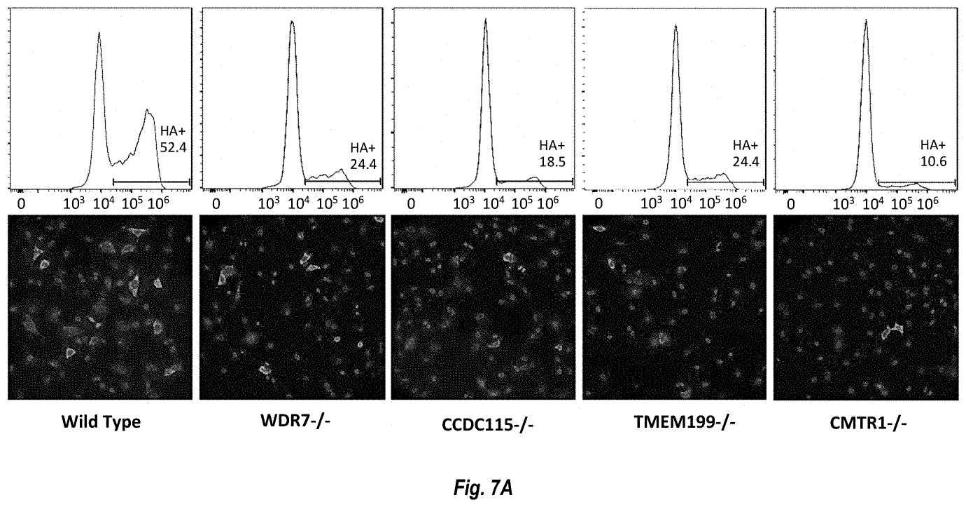

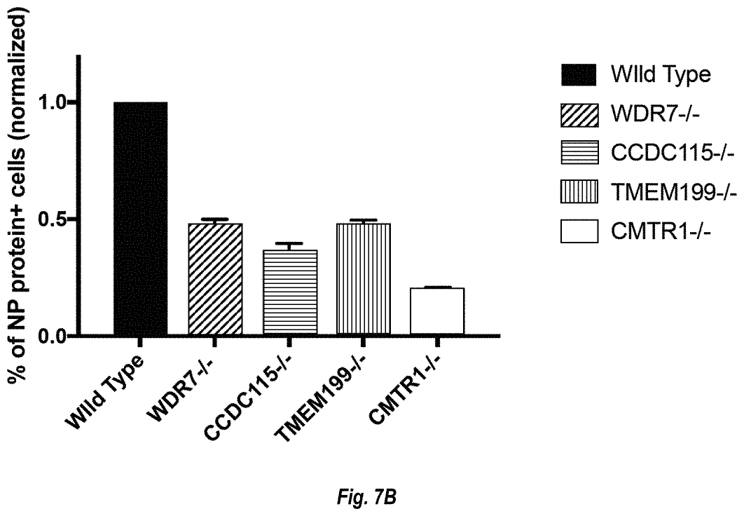

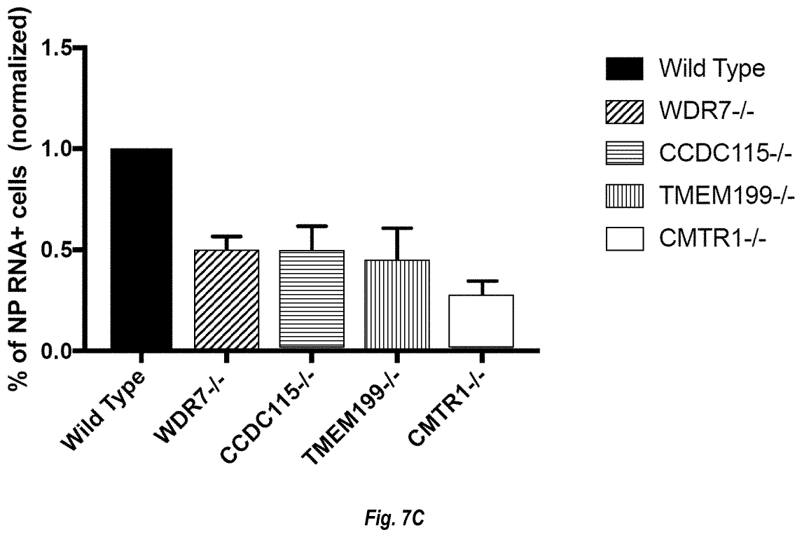

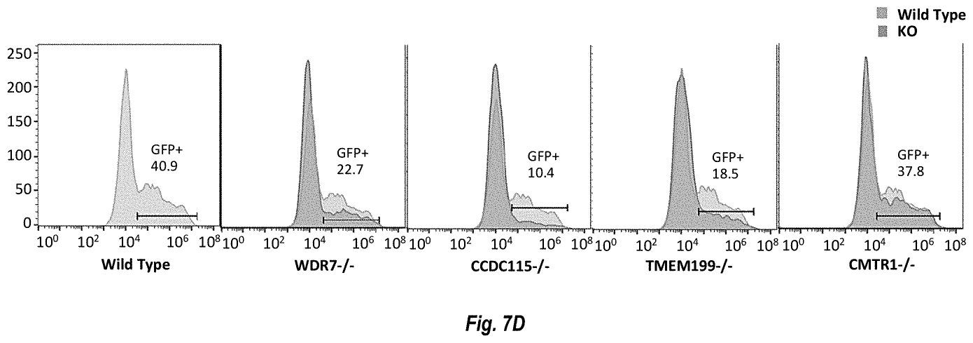

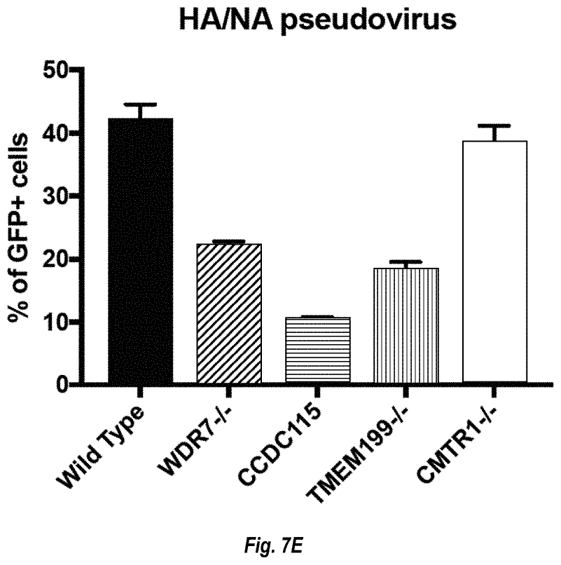

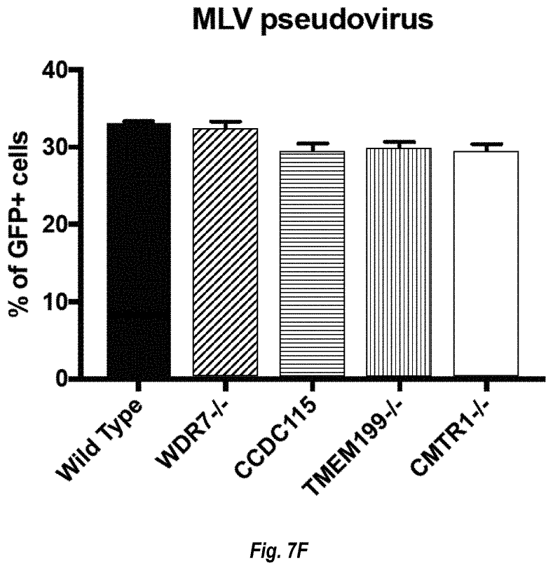

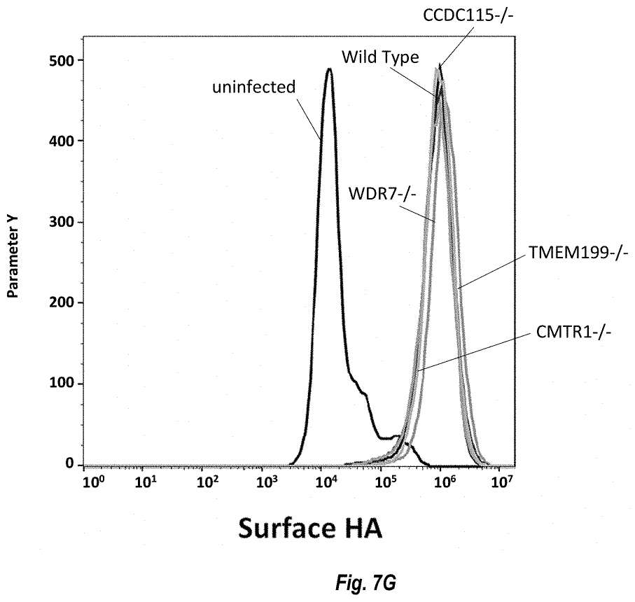

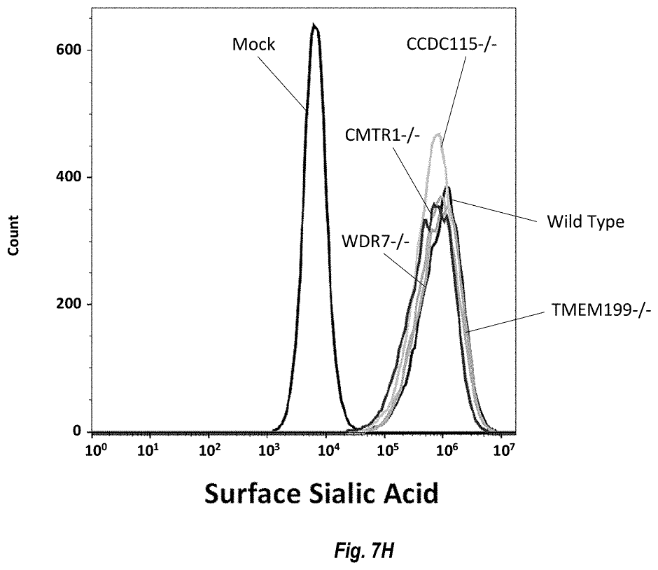

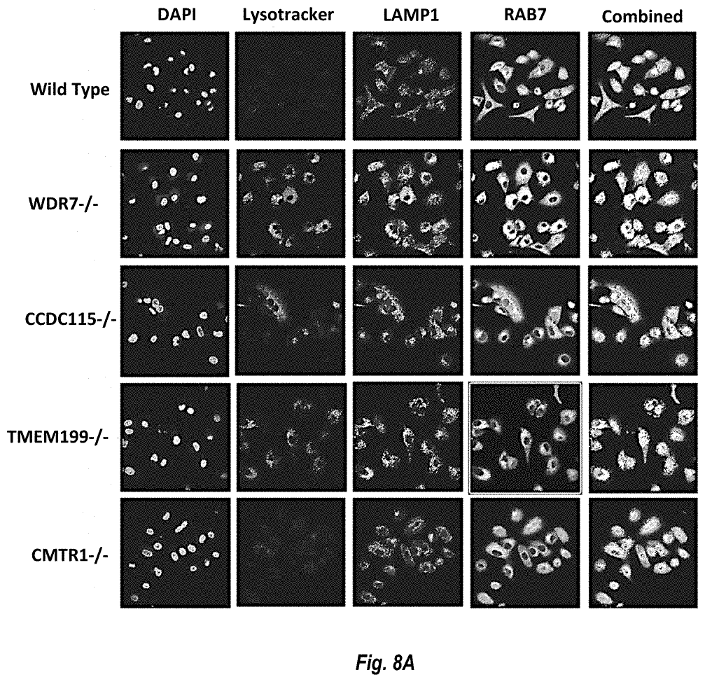

To better understand how loss of WDR7, CCDC115, TMEM199 and CMTR1 confer resistance against IAV infection, which steps of the IAV life cycle they are involved in was determined. Significant reduction in viral nucleoprotein (NP) RNA and protein levels was found at 4 hours post-infection in KO A549 cells compared to wild type, suggesting that all 4 genes are important for an early step in IAV infection (FIG. 7A). (Rimmelzwaan G F, et al. J Virol Methods. 1998; 74(1):57-66.) To test if the genes are required for IAV entry, KO and wild type cells were transduced with a MLV-GFP retrovirus that had been pseudotyped with IAV H1N1 envelop HA and NA. This allows the retrovirus to enter the cell in a HA-dependent manner that is akin to IAV entry. (Huang et al, J Virol. 2008; 82(10):4834-43). GFP expression was then monitored in the cells 48 hours post-transduction. It was found that WDR7-, CCDC115- and TMEM199-KO cells but not CMTR-1 KO cells have lower percentage of GFP-expressing cells compared to wild type (FIG. 7D). Interestingly, all the KO cells have comparable GFP expression to wild type cells when transduced with a MLV-GFP retrovirus that has been pseudotyped with MLV-envelop protein, suggesting that WDR7, CCDC115 and TMEM199 are specifically required for IAV entry in a HA/NA dependent manner. It was next determined whether the three genes are required for IAV entry by allowing virus attachment to the cell membrane. To test this, KO and wild type cells were incubated with PR8 virus at 4° C. for 30 minutes and stained for surface HA. No difference in HA staining between KO and wild type cells was found, suggesting that the genes are not essential for virus attachment (FIG. 7G). This is also supported by the observation that WDR7, CCDC115, and TMEM199 do not affect expression of cell surface sialic acid (FIG. 7G), which serve as entry receptor for IAV. (Luo M. Adv Exp Med Biol. 2012; 726:201-21.)

### Example 5: WDR7, CCDC115 and TMEM199 Regulate Endo-Lysosomal Acidification

Recent studies have reported WDR7, CCDC115 and TMEM199 as accessory proteins to the mammalian V-type ATPase. (Merkulova M, et al. Sci Rep. 2005; 5:14827; Miles A L, et at. Elife. 2017; 6:e22693.) In addition, WDR7 has been shown to interact with the mammalian homologue (DMXL2) (Sethi et al. J Biol Chem. 2010; 285(45):34757-64) of the yeast RAVE complex responsible for regulating assembly and disassembly of V-type ATPases (Smardon et al. J Biol Chem. 2002; 277(16): 13831-9). CCDC115 and TMEM199 have also been reported as orthologues of the Yeast V-type ATPase assembly factors Vma22p and Vma12p (Jansen et al. Am J hum Genet. 2016; 98(2):310-21; Jansen et al. Am J Hum Genet. 2016; 98(2): 322-30), which are required for V-type ATPase assembly in the endoplasmic reticulum (ER) (Graham et al. J Cell Biol. 1998; 142(1):39-49).

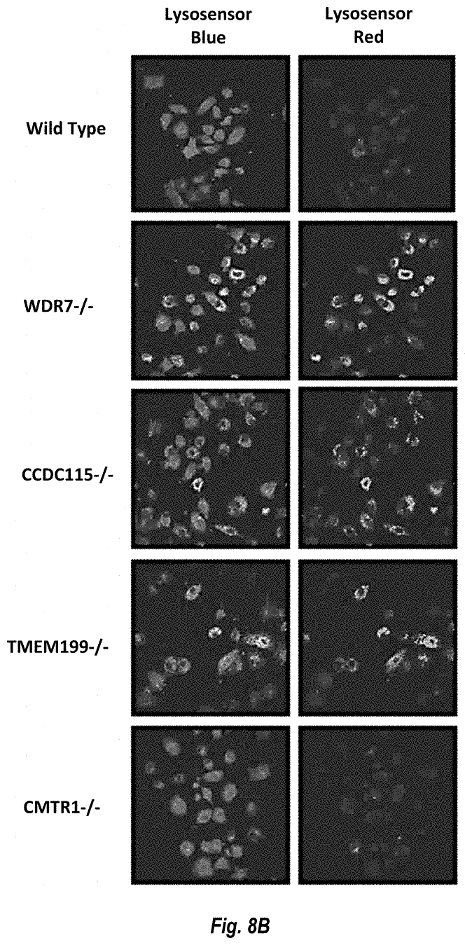

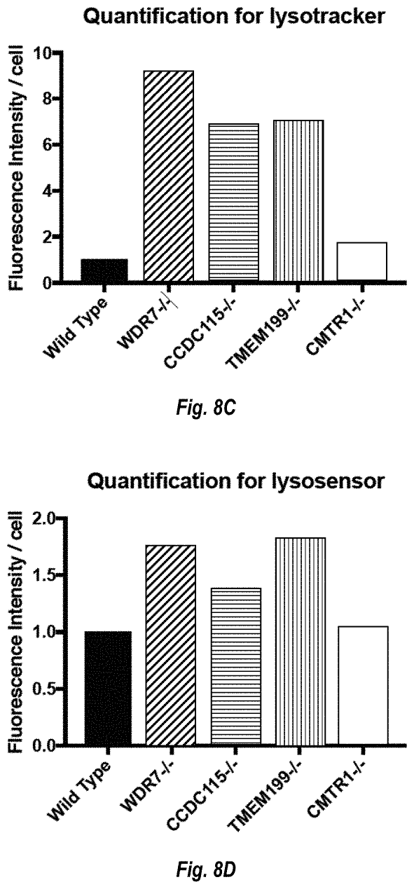

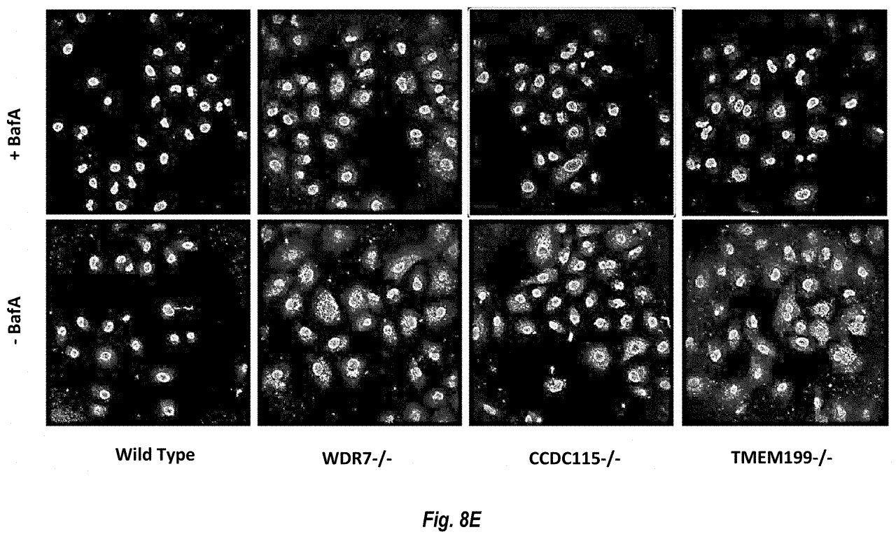

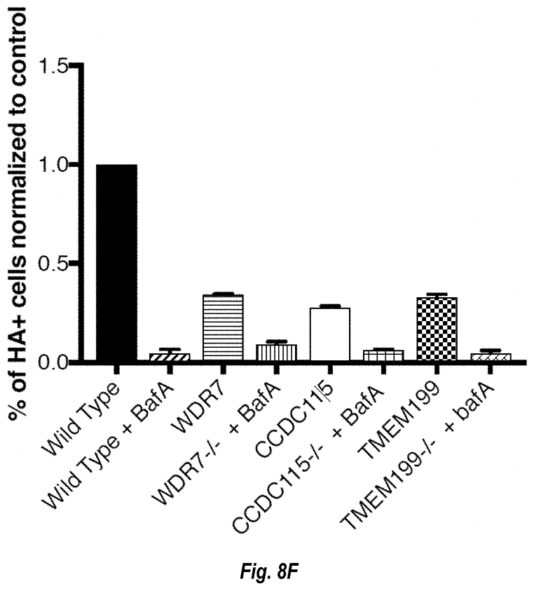

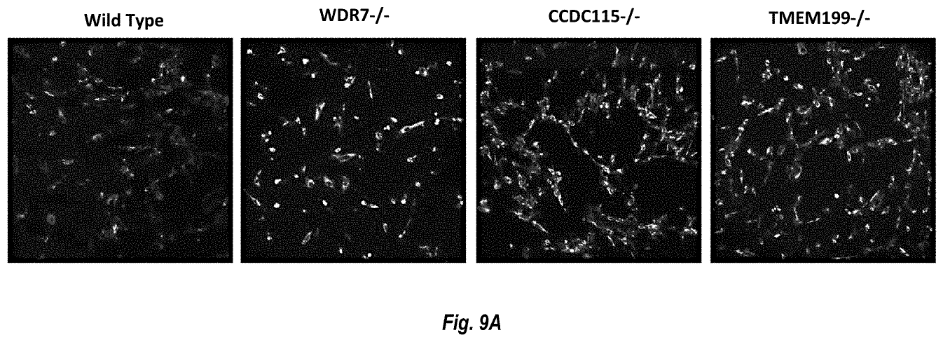

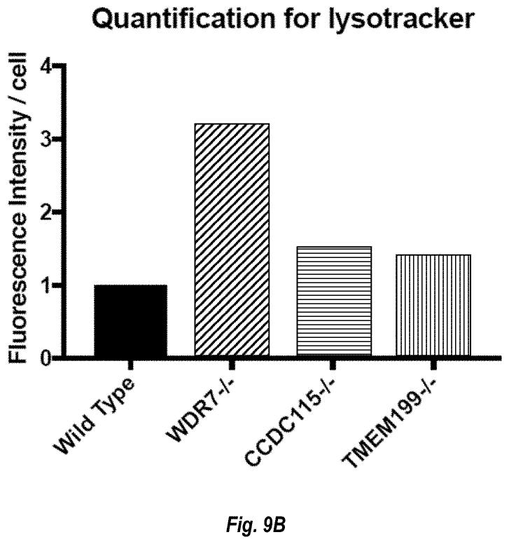

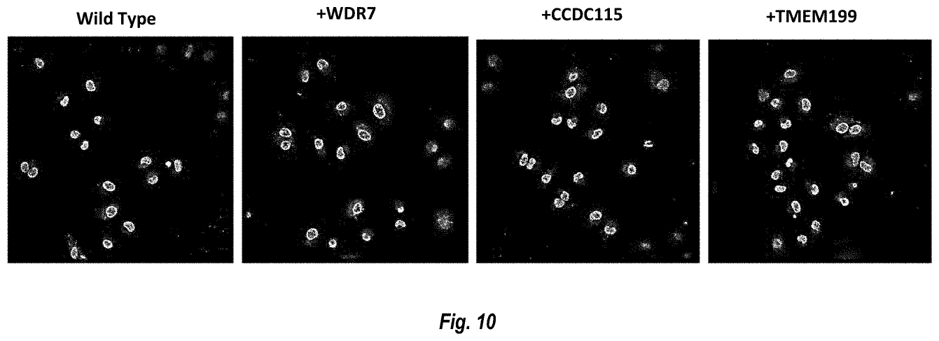

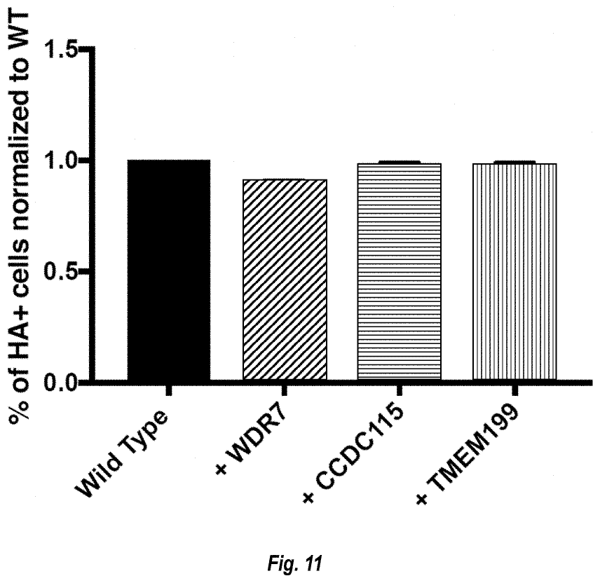

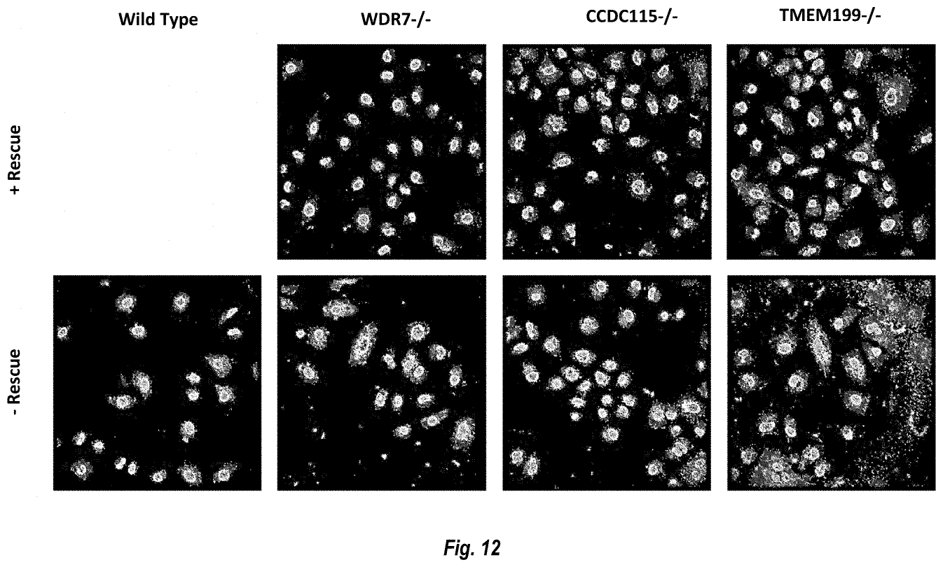

To test if WDR7, CCDC115 and TMEM199 are required for IAV entry by facilitating endo-lysosomal acidification, KO and wild type A549 cells were stained using lysotracker red, fluorescent-labelled anti-Rab7 and anti-LAMP1 antibodies. Unexpectedly, an increase in lysotracker red staining was observed in WDR7-, CCDC115- and TMEM-KO cells, but not in CMTR1-KO or wild type cells, suggesting that the former negatively regulate V-type ATPase activity (FIG. 8A). An increase in lysotracker red staining was also observed in HLFs cells lacking WDR7, CCDC115 or TMEM199 (FIG. 9). This is in contrast to a previous study which showed that CCDC115 and TMEM199 are required for endosomal acidification in HELA cells. (Miles A L, et at. Elife. 2017; 6:e22693.) The difference in phenotype may be cell type-dependent. Indeed, while deletion of WDR7, CCDC115 and TMEM199 increased lysotracker staining in A549 and HLF cells, a clear difference was not observed in HELA cells (data not shown). Interestingly, ectopic expression of WDR7, CCDC115 or TMEM199 in wild type A549 cells did not have an effect on lysotracker staining or IAV infection (FIG. 10, FIG. 11), but re-expression of sgRNA-resistant copies of the genes in the KO cells restored normal levels of lysotracker staining (FIG. 12). This led to the hypothesis that WDR7, CCDC115 and TMEM199 regulate V-type ATPase activity in a dynamic manner rather than simply acting as inhibitors.

The lysotracker red staining in the KO cells overlapped partly with Rab7 (late endosome) and LAMP1 (lysosome). To determine if the increase in lysotracker signal is due to expansion of the endo-lysosomal compartments or actual reduction in pH, WDR7-, CCDC115- and TMEM199-KO cells were stained with the more pH-sensitive lysosensor blue dye. As in the case with lysotracker red, an increase in lysosensor blue staining was observed in WDR7-, CCDC115 and TMEM199-KO cells, indicating a reduction in endo-lysosomal pH (FIG. 8B).

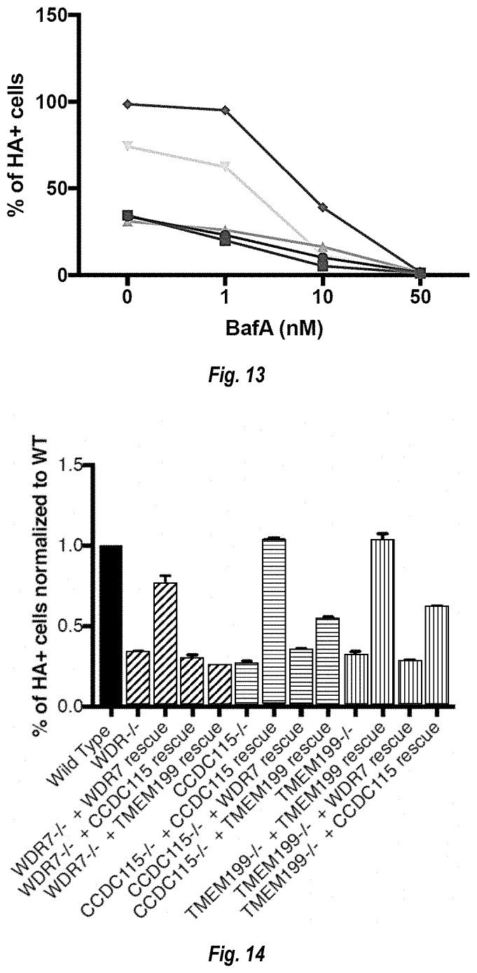

To further probe their roles in endo-lysosomal acidification, WDR7-, CCDC115- and TMEM199-KO cells were treated with Bafilomycin A (BafA), which has been known to inhibit V-type ATPase activity and block IAV infection. (Yoshimori T, et al. J Biol Chem. 1991; 266(26):17707-12; Ochiai H, et al. Antiviral Res. 1995; 27(4):425-30.) As expected, a decrease in lysotracker staining was observed in both wild type and KO cells that were treated with BafA (FIG. 8E). However, BafA treatment, even at low concentrations, further protected the KO cells against IAV infection (FIG. 8F, FIG. 13), suggesting that fine tuning of ATPase activity is required for efficient infection. WDR7, CCDC115 and TMEM199 also appeared to play non-redundant roles as over-expression of sgRNA-resistant WDR7 gene into CCDC115- or TMEM199-KO cells and vice versa did not rescue IAV infection (FIG. 14).

### Example 6: Loss of WDR7, CCDC115 and TMEM199 Prevents IAV Entry into the Nucleus and Increases Lysosomal Degradation of Viral RNA

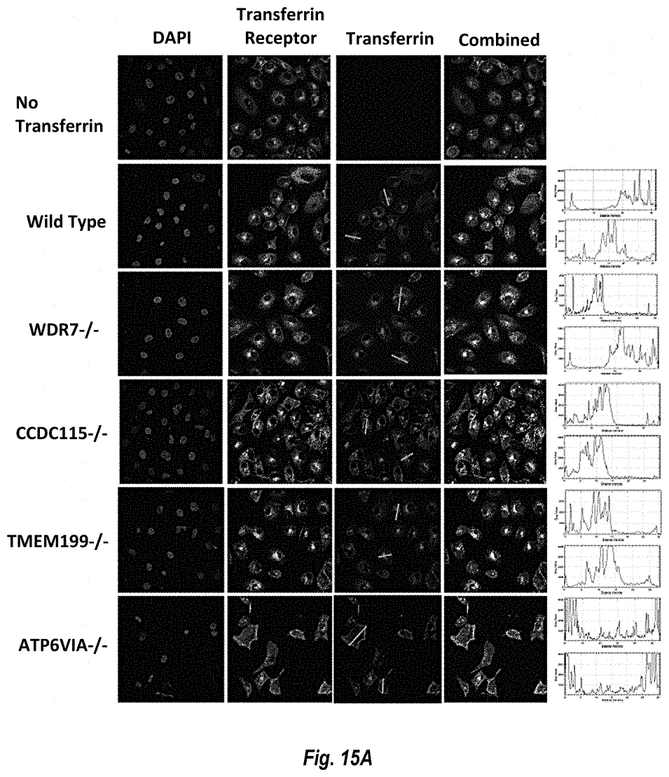

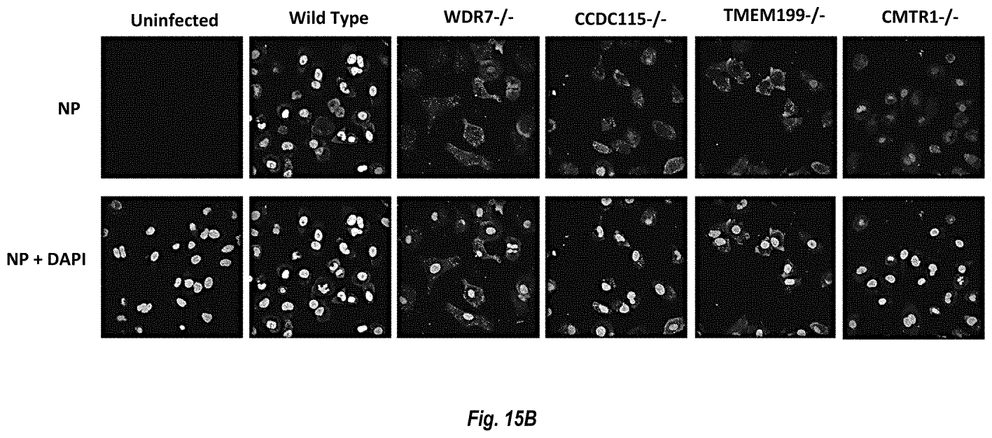

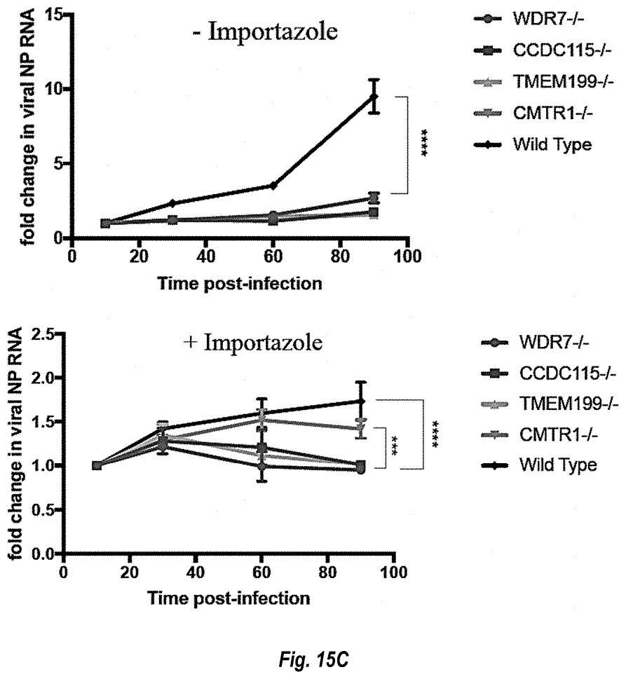

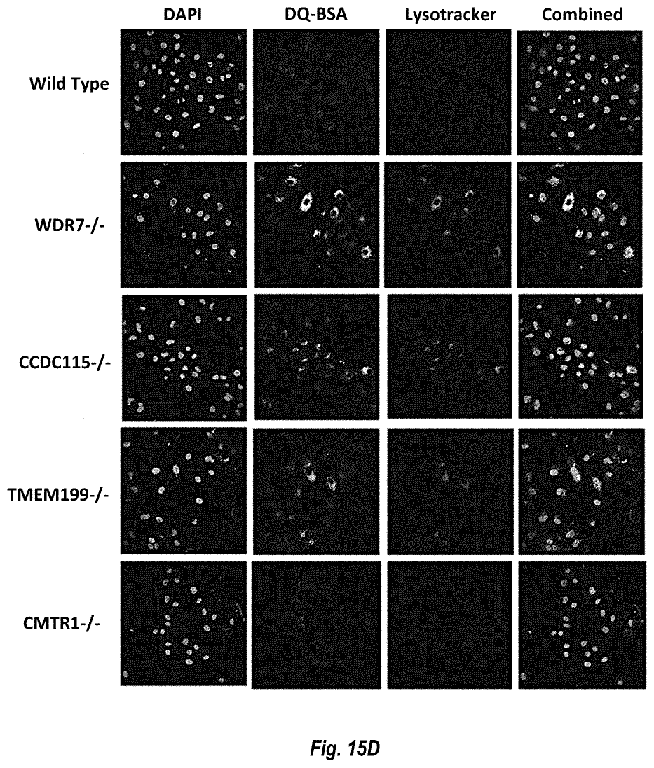

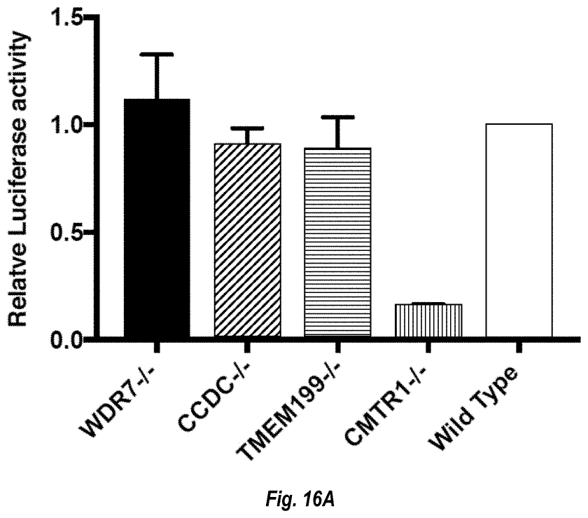

It was next determined how perturbation of the V-type ATPases and reduction in endosomal pH in WDR7-, CCDC115- and TMEM199-KO cells block IAV infection. Studies have reported that loss of V-type ATPase impairs clathrin-mediated endocytosis, which IAV may utilize to enter the cell. (Kozik P, et al. Nat Cell Biol. 2013; 15(1):50-60; Matlin K S, et al. J Cell Biol. 1981; 91:601-13.) To test if WDR7, CCDC115 and TMEM199 are required for clathrin-mediated endocytosis, KO and wild type cells were incubated with fluorescent-labelled transferrin at 37° C. for 60 minutes. A reduction in transferrin uptake was observed in ATP6V1A-KO cells but not WDR7-, CCDC115- and TMEM199-KO cells, suggesting that the block in infection occurs downstream of endocytosis (FIG. 15A). It was then determined whether WDR7, CCDC115 and TMEM199 impact IAV localization within the cell upon infection. KO and wild type cells were infected at MOI 200 and stained for intracellular Influenza NP protein at 2 hours post-infection. In wild type cells, NP staining is primarily observed in the nuclei, where viral replication takes place. In contrast, NP staining is largely absent in the nuclei of KO cells, and is instead concentrated in specks at the surface and in the pen-nuclear regions (FIG. 15B). Taken together, these data suggest that IAV infection is likely blocked after endocytosis but prior to nuclear entry.

During IAV infection, gradual exposure to acidic pH as the virus moves from early to late endosome triggers a conformational change in the HA protein leading to fusion of the viral and endosome membrane. (Kozik P, et al. Nat Cell Biol. 2013; 15(1):50-60; Matlin K S, et al. J Cell Biol. 1981; 91:601-13.) Although an acidic endo-lysosomal environment is required for viral fusion, studies have shown that exposure to pH lower than the optimal fusion pH may cause HA inactivation and coagulation of viral ribonucleoproteins (RNP). (Costello D A, et al. J Virol. 2015; 89(1): 350-60; Fontana J, et al. J Virol. 2012; 86(6):2919-29.) In addition, perturbation of V-type ATPase activity and localization can disrupt the pH gradient from early to late endosomes which IAV requires for efficient uncoating. (Huotari J, Helenius A. EMBO J. 2011; 30(17): 3481-3500; Li S, et al. Biophys J. 2014; 106(7): 1447-56.)

It was thus hypothesized that the reduction in endo-lysosomal pH in WDR7-, CCDC115- and TMEM199-KO cells blocks IAV fusion and uncoating, which causes majority of the incoming virus to be degraded within the lysosomes. To test this, IAV NP RNA degradation was measured in KO and wild type cells up to 90 minutes post-infection by qRT-PCR. To remove the effect of viral transcription and replication, cells were pre-treated with 40 uM Importazole, which has been reported to block IAV transport into the nucleus. (Chou Y Y, et al. PLoS Pathog. 2013; 9(5):e1003359.) A slight increase in viral RNA level was observed from 10 to 30 minutes post-infection in both KO and wild type cells, which may represent the time taken for internalization of all the bound virions. Viral RNA level then declines from 30 to 90 minutes in WDR7-, CCDC115- and TMEM199-KO cells but not in CMTR1-KO or wild type cells (FIG. 15C). Interestingly, a general increase in lysosomal degradation of endocytic cargo (DQ-BSA) was observed in WDR7-, CCDC115- and TMEM199-KO cells, suggesting that the increase in IAV degradation may be partly due to increased endo-lysosome trafficking (FIG. 15D). (Murrow L, et al. Nat Cell Biol. 2015; 17(3):300-10.)

### Example 7: Loss of CMTR1 Inhibits Viral Replication and Increases Expression of Anti-Viral Genes

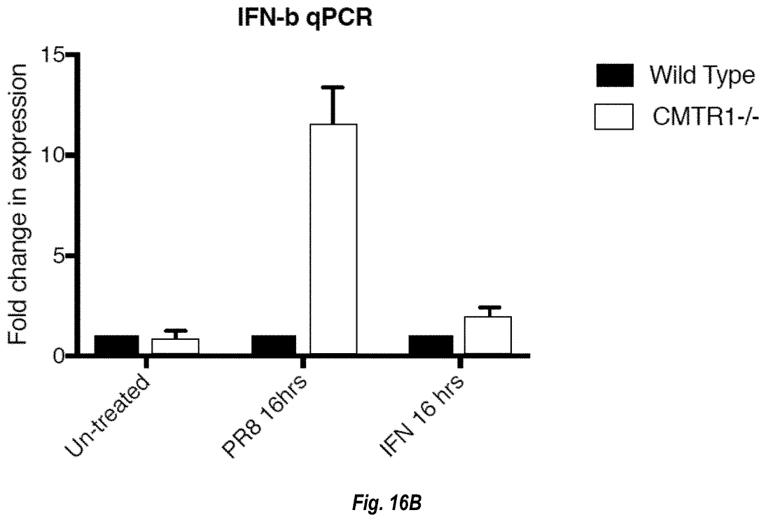

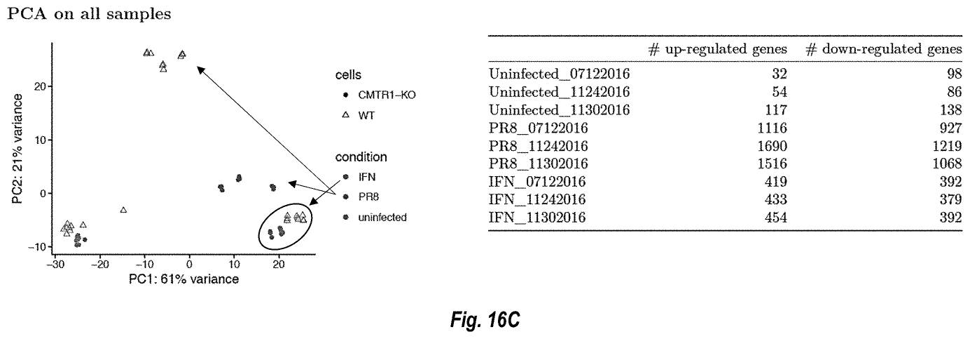

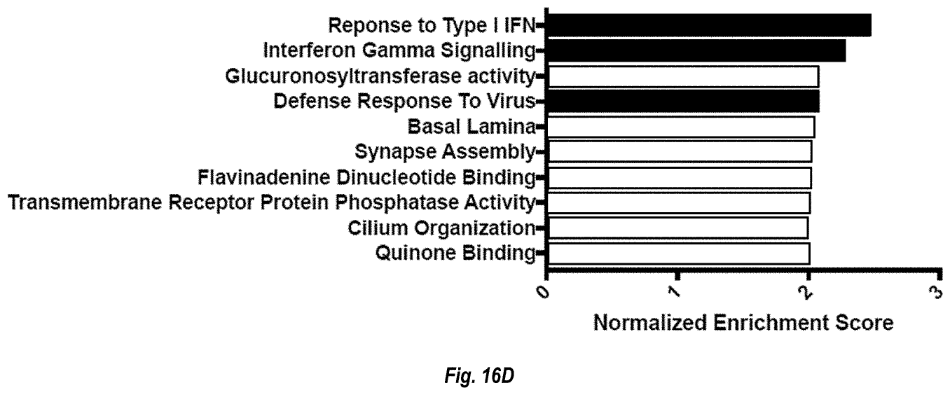

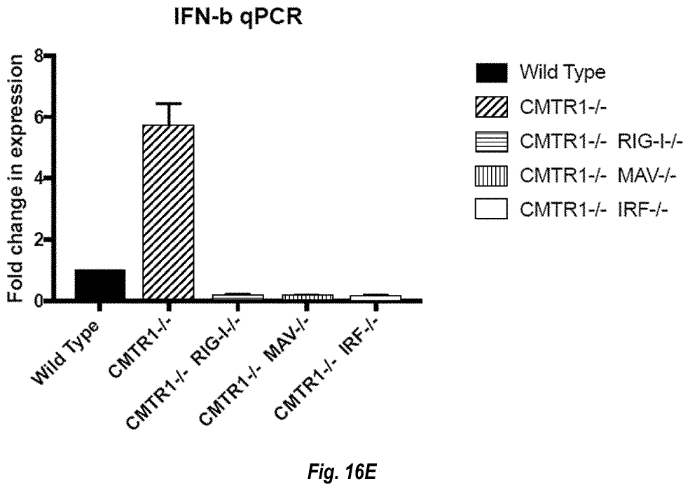

CMTR1 was recently discovered as the human 2′-O-ribose cap methyltransferase, which adds a methyl-group to the 5′-7 methylguanosine cap of eukaryotic mRNA to form the Cap1 structure. (Belanger F, et al. J Biol Chem. 2010; 285(43):33037-44; Smietanski M, et al. Nature Commun. 2014; 5:3004.) Since 2′-O-methylation of the mRNA cap has been known to be important for IAV cap snatching, it was hypothesized that the loss of CMTR1 would inhibit viral transcription. (Bouloy M, et al. Proc Natl Acad Sci USA. 1980; 77(7):3952-6; Wakai C, et al. J Virol. 2011; 85(15):7504-12.) To test this, KO and control cells were transfected with a vRNA-luciferase reporter as well as plasmids expressing PR8 polymerase subunits PA, PB1 and PB2. (Lutz A, et al. J Virol Methods. 2005; 126(1-2):13-20.) 24 hours post-transfection, cells were lysed and Luciferase/Renilla activity was measured. As expected, lower luciferase activity was observed in CMTR1-KO cells but not in WDR7-, CCDC115-, TMEM199-KO cells or wild type cells (FIG. 16A).

Although the Cap1 structure is present on most eukaryotic mRNA, its precise functions are poorly understood as the lack of CMTR1 does not seem to have a significant impact on global protein translation. (Belanger F, et al. J Biol Chem. 2010; 285(43):33037-44) Recent studies have reported that 2-O-ribose methylation of the mRNA cap acts as a mechanism by which the cell differentiates between self- and non-self RNA, and siRNA knockdown of CMTR1 was shown to elevate Type I IFN response in A549 cells. (Zust R, et al. Nat Immunol. 2011; 12(2): 137-43; Schuberth-Wanger C, et al. Immunity. 2015; 43(1):41-51.) Coronavirus and Flavivirus are also known to encode their own 2′-O-methyltransferase, the absence of which leads to elevated type I IFN production during infection. (Dong H, et al. Antiviral Res. 2008; 80(1):1-10; Menachery V D, et al. J Virol. 2014; 88(8):4251-64.) It was thus hypothesized that the loss of CMTR1 blocks IAV infection by both preventing efficient cap snatching and increasing cellular antiviral response. To test this, IFN-β levels were measured in control and CMTR1-KO cells in the presence and absence of PR8 infection via qRT-PCR. Interestingly, an increase in IFN-β expression was observed in CMTR1-KO cells but only when they were infected by PR8 virus (FIG. 16B). To confirm those results, RNA was extracted from CMTR1-KO and wild type cells with and without PR8 infection and performed RNA sequencing. A large number of differentially expressed genes between CMTR1-KO and wild type cells were found in the presence of PR8 infection but not at the resting state (FIG. 16C, FIG. 16D). A closer inspection of these genes revealed significant enrichment of Type I and II IFN-related genes as well as other antiviral genes, in agreement with the qRT-PCR results. To test if the increase in IFN signature is mediated by the RIG-I sensing pathway, CMTR1-KO cells were transduced with sgRNA targeting RIG-I, MAV or IRF3. (Loo Y M, Gale M Jr. Immunity. 2011; 34(5):680-92.) It was found that the increase in IFN-β expression is completely abrogated in the absence of RIG-I, MAV or IRF3, indicating that an intact RNA sensing pathway is required for the elevated IFN response in CMTR1-KO cells (FIG. 16E). Given the lack of cytoxicity and its dual effect on IAV infection, CMTR1 is a good target for therapeutic interventions.

### Example 8: Loss of CMTR1 Synergizes with Baloxavir Marboxil to Protect Against IAV Infection

As note herein, CMTR1 is necessary for 2-O-ribose methylation of the mRNA cap. This cap modification is required by the Influenza A virus endonuclease PA subunit to efficiently cleave the host mRNA cap and carry out cap-snatching, a step that is important for viral replication and translation. The recently FDA-approved anti-Influenza drug Xofluza® (baloxavir marboxil) acts in the same pathway as CMTR1 by directly inhibiting the endonuclease activity of the Influenza virus polymerase PA subunit. It was therefore hypothesized that targeting CMTR1 in combination with Xofluza treatment may trigger synergistic protection against Influenza A virus infection.

HA+ A549 wild-type, WDR7-KO, CCDC115-KO, TMEM199-KO, and CMTR1-KO cells were treated with 1 nM, 5 nM, or 10 nM baloxavir marboxil for 30 minutes at 37° C., and then infected with Influenza A PR8 virus at MOI 5 for 16 hours. Baloxavir marboxil was also added during the infection and post-infection. Cells were then harvested and stained for surface Influenza Hemagglutinin.

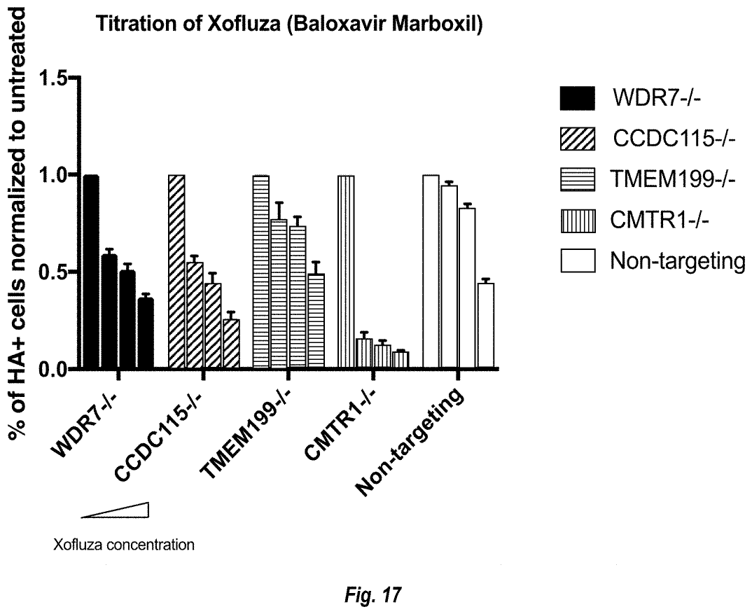

The results are shown in FIG. 17. In wild type cells, treatment with 1 nM, 5 nM, and 10 nM of baloxavir marboxil reduced the rate of Influenza A virus infection by 6%, 19%, and 58%, respectively, compared to untreated cells, according to Hemagglutinin staining. In contrast, treatment of CMTR1−/− (CMTR1-KO) cells with similar concentrations of baloxavir marboxil reduced the rate of infection by 87%, 89%, and 91%, respectively. This suggests that deletion of CMTR1 in conjunction with baloxavir marboxil treatment confers synergistic protection against Influenza A virus infection. CRISPR deletion of WDR7, CCDC115 and TMEM199 also exhibited synergistic effect with baloxavir marboxil treatment but to a lesser extent compared to CMTR1. Combination treatment of baloxavir marboxil with a CMTR1 inhibitor would be expected to be significantly more effective for preventing IAV infection than baloxavir marboxil alone. Combination treatment of baloxavir marboxil with a WDR7 inhibitor, a CCDC115 inhibitor, or a TMEM199 inhibitor would also be expected to be more effective for preventing IAV infection than baloxavir marboxil alone.

The foregoing written specification should enable one skilled in the art to practice embodiments within the scope of the appended claims. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

TABLE OF CERTAIN SEQUENCES

| SEQ | | |
|---|---|---|
| ID | Descrip- | |
| NO | tion | Sequence |
| 7 | Human | MAGNSLVLPI VLWGRKAPTH CISAVLLTDD |
| | WDR7 | GATIVTGCHD GQICLWDLSV ELQINPRALL |
| | (NCBI | FGHTASITCL SKACASSDKQ YIVSASESGE |
| | Ref. NP_ | MCLWDVSDGR CIEFTKLACT HTGIQFYQFS |
| | 056100.2) | VGNQREGRLL CHGHYPEILV VDATSLEVLY |
| | | SLVSKISPDW ISSMSIIRSH RTQEDTVVAL |
| | | SVTGILKVWI VTSEISDMQD TEPIFEEESK |
| | | PIYCQNCQSI SFCAFTQRSL LVVCSKYWRV |
| | | FDAGDYSLLC SGPSENGQTW TGGDFVSSDK |
| | | VIIWTENGQS YIYKLPASCL PASDSFRSDV |
| | | GKAVENLIPP VQHILLDRKD KELLICPPVT |
| | | RFFYGCREYF HKLLIQGDSS GRLNIWNISD |
| | | TADKQGSEEG LAMTTSISLQ EAFDKLNPCP |
| | | AGIIDQLSVI PNSNEPLKVT ASVYIPAHGR |
| | | LVCGREDGSI VIVPATQTAI VQLLQGEHML |
| | | RRGWPPHRTL RGHRNKVTCL LYPHQVSARY |
| | | DQRYLISGGV DFSVIIWDIF SGEMKHIFCV |
| | | HGGEITQLLV PPENCSARVQ HCICSVASDH |
| | | SVGLLSLREK KCIMLASRHL FPIQVIKWRP |
| | | SDDYLVVGCS DGSVYVWQMD TGALDRCVMG |
| | | ITAVEILNAC DEAVPAAVDS LSHPAVNLKQ |
| | | AMTRRSLAAL KNMAHHKLQT LATNLLASEA |
| | | SDKGNLPKYS HNSLMVQAIK TNLTDPDIHV |
| | | LFFDVEALII QLLTEEASRP NTALISPENL |
| | | QKASGSSDKG GSFLTGKRAA VLFQQVKETI |
| | | KENIKEHLLD DEEEDEEIMR QRREESDPEY |
| | | RSSKSKPLTL LEYNLTMDTA KLFMSCLHAW |
| | | GLNEVLDEVC LDRLGMLKPH CTVSFGLLSR |
| | | GGHMSLMLPG YNQPACKLSH GKTEVGRKLP |
| | | ASEGVGKGTY GVSRAVTTQH LLSIISLANT |
| | | LMSMTNATFI GDHMKKGPTR PPRPSTPDLS |
| | | KARGSPPTSS NIVQGQIKQV AAPVVSARSD |
| | | ADHSGSDPPS APALHTCFLV NEGWSQLAAM |
| | | HCVMLPDLLG LDKFRPPLLE MLARRWQDRC |
| | | LEVREAAQAL LLAELRRIEQ AGRKEAIDAW |
| | | APYLPQYIDH VISPGVTSEA AQTITTAPDA |
| | | SGPEAKVQEE EHDLVDDDIT TGCLSSVPQM |
| | | KKISTSYEER RKQATAIVLL GVIGAEFGAE |
| | | IEPPKLLTRP RSSSQIPEGF GLTSGGSNYS |
| | | LARHTCKALT FLLLQPPSPK LPPHSTIRRT |
| | | AIDLIGRGFT VWEPYMDVSA VLMGLLELCA |
| | | DAEKQLANIT MGLPLSPAAD SARSARHALS |
| | | LIATARPPAF ITTIAKEVHR HTALAANTQS |
| | | QQNMHTTTLA RAKGEILRVI EILIEKMPTD |
| | | VVDLLVEVMD IIMYCLEGSL VKKKGLQECF |
| | | PAICRFYMVS YYERNHRIAV GARHGSVALY |
| | | DIRTGKCQTI HGHKGPITAV AFAPDGRYLA |
| | | TYSNTDSHIS FWQMNTSLLG SIGMLNSAPQ |
| | | LRCIKTYQVP PVQPASPGSH NALKLARLIW |
| | | TSNRNVILMA HDGKEHRFMV |
| 8 | Human | MAALDLRAEL DSLVLQLLGD LEELEGKRTV |
| | CCDC115 | LNARVEEGWL SLAKARYAMG AKSVGPLQYA |
| | (NCBI | SHMEPQVCLH ASEAQEGLQK FKVVRAGVHA |
| | Ref. NP_ | PEEVGPREAG LRRRKGPTKT PEPESSEAPQ |
| | 115733.2) | DPLNWFGILV PHSLRQAQAS FRDGLQLAAD |
| | | IASLQNRIDW GRSQLRGLQE KLKQLEPGAA |
| 9 | Human | MASSLLAGER LVRALGPGGE LEPERLPRKL |
| | TMEM199 | RAELEAALGK KHKGGDSSSG PQRLVSFRLI |
| | (NCBI | RDLHQHLRER DSKLYLHELL EGSEIYLPEV |
| | Ref. NP_ | VKPPRNPELV ARLEKIKIQL ANEEYKRITR |
| | 689677.1) | NVTCQDTRHG GTLSDLGKQV RSLKALVITI |
| | | FNFIVTVVAA FVCTYLGSQY IFTEMASRVL |
| | | AALIVASVVG LAELYVMVRA MEGELGEL |
| 10 | Human | MKRRTDPECT APIKKQKKRV AELALSLSST |
| | CMTR1 | SDDEPPSSVS HGAKASTTSL SGSDSETEGK |
| | (NCBI | QHSSDSFDDA FKADSLVEGT SSRYSMYNSV |
| | Ref. NP_ | SQKLMAKMGF REGEGLGKYS QGRKDIVEAS |
| | 055865.1) | SQKGRRGLGL TLRGFDQELN VDWRDEPEPS |
| | | ACEQVSWFPE CTTEIPDTQE MSDWMVVGKR |
| | | KMIIEDETEF CGEELLHSVL QCKSVFDVLD |
| | | GEEMRRARTR ANPYEMIRGV FFLNRAAMKM |
| | | ANMDFVFDRM FTNPRDSYGK PLVKDREAEL |
| | | LYFADVCAGP GGFSEYVLWR KKWHAKGFGM |
| | | TLKGPNDFKL EDFYSASSEL FEPYYGEGGI |
| | | DGDGDITRPE NISAFRNFVL DNTDRKGVHF |
| | | LMADGGFSVE GQENLQEILS KQLLLCQFLM |
| | | ALSIVRTGGH FICKTFDLFT PFSVGLVYLL |
| | | YCCFERVCLF KPITSRPANS ERYVVCKGLK |
| | | VGIDDVRDYL FAVNIKLNQL RNTDSDVNLV |
| | | VPLEVIKGDH EFTDYMIRSN ESHCSLQIKA |
| | | LAKIHAFVQD TTLSEPRQAE IRKECLRLWG |
| | | IPDQARVAPS SSDPKSKFFE LIQGTEIDIF |
| | | SYKPTLLTSK TLEKIRPVFD YRCMVSGSEQ |
| | | KFLIGLGKSQ IYTWDGRQSD RWIKLDLKTE |
| | | LPRDTLLSVE IVHELKGEGK AQRKISAIHI |
| | | LDVLVLNGTD VREQHFNQRI QLAEKFVKAV |
| | | SKPSRPDMNP IRVKEVYRLE EMEKIFVRLE |
| | | MKIIKGSSGT PKLSYTGRDD RHFVPMGLYI |
| | | VRTVNEPWTM GFSKSFKKKF FYNKKTKDST |
| | | FDLPADSIAP FHICYYGRLF WEWGDGIRVH |
| | | DSQKPQDQDK LSKEDVLSFI QMHRA |

What is claimed is:

1. A method of treating influenza A virus infection comprising administering to a subject in need thereof an effective amount of at least one agent selected from a WDR7 inhibitor, a CCDC115 inhibitor, a TMEM199 inhibitor, and a CMTR1 inhibitor.

2. The method of claim 1, wherein at least one agent is a WDR7 inhibitor.

3. The method of claim 2, wherein the WDR7 inhibitor is selected from an antibody, an antisense oligonucleotide, an siRNA, a peptide, an abzyme, or a small molecule.

4. The method of claim 3, wherein the WDR7 inhibitor is a small molecule.

5. The method of claim 3, wherein the WDR7 inhibitor is an antisense oligonucleotide or an siRNA.

6. The method of claim 5, wherein the antisense oligonucleotide is complementary to a portion of the WDR7 mRNA.

7. The method of claim 3, wherein the WDR7 inhibitor is a peptide.

8. The method of claim 3, wherein the WDR7 inhibitor is an antibody.

9. The method of claim 8, wherein the antibody is an antibody fragment.

10. The method of claim 9, wherein the antibody fragment is selected from an scFv, Fab, Fab′, F(ab′)2 fragment.

11. The method of any one of the preceding claims, wherein at least one agent is a CCDC115 inhibitor.

12. The method of claim 11, wherein the CCDC115 inhibitor is selected from an antibody, an antisense oligonucleotide, an siRNA, a peptide, an abzyme, or a small molecule.

13. The method of claim 11, wherein the CCDC115 inhibitor is a small molecule.

14. The method of claim 11, wherein the CCDC115 inhibitor is an antisense oligonucleotide or an siRNA.

15. The method of claim 14, wherein the antisense oligonucleotide is complementary to a portion of the CCDC115 mRNA.

16. The method of claim 11, wherein the CCDC115 inhibitor is a peptide.

17. The method of claim 11, wherein the CCDC115 inhibitor is an antibody.

18. The method of claim 17, wherein the antibody is an antibody fragment.

19. The method of claim 18, wherein the antibody fragment is selected from an scFv, Fab, Fab′, F(ab′)2 fragment.

20. The method of any one of the preceding claims, wherein at least one agent is a TMEM199 inhibitor.

21. The method of claim 20, wherein the TMEM199 inhibitor is selected from an antibody, an antisense oligonucleotide, an siRNA, a peptide, an abzyme, or a small molecule.

22. The method of claim 20, wherein the TMEM199 inhibitor is a small molecule.

23. The method of claim 20, wherein the TMEM199 inhibitor is an antisense oligonucleotide or an siRNA.

24. The method of claim 23, wherein the antisense oligonucleotide is complementary to a portion of the TMEM199 mRNA.

25. The method of claim 20, wherein the TMEM199 inhibitor is a peptide.

26. The method of claim 20, wherein the TMEM199 inhibitor is an antibody.

27. The method of claim 26, wherein the antibody is an antibody fragment.

28. The method of claim 27, wherein the antibody fragment is selected from an scFv, Fab, Fab′, F(ab′)2 fragment.

29. The method of any one of the preceding claims, wherein at least one agent is a CMTR1 inhibitor.

30. The method of claim 29, wherein the CMTR1 inhibitor is selected from an antibody, an antisense oligonucleotide, an siRNA, a peptide, an abzyme, or a small molecule.

31. The method of claim 30, wherein the CMTR1 inhibitor is a small molecule.

32. The method of claim 30, wherein the CMTR1 inhibitor is an antisense oligonucleotide or an siRNA.

33. The method of claim 32, wherein the antisense oligonucleotide is complementary to a portion of the CMTR1 mRNA.

34. The method of claim 30, wherein the CMTR1 inhibitor is a peptide.

35. The method of claim 30, wherein the CMTR1 inhibitor is an antibody.

36. The method of claim 35, wherein the antibody is an antibody fragment.

37. The method of claim 36, wherein the antibody fragment is selected from an scFv, Fab, Fab′, F(ab′)2 fragment.

38. The method of any one of the preceding claims, wherein the agent reduces nuclear entry of influenza A.

39. The method of any one of the preceding claims, wherein the agent reduces viral replication.

40. The method of claim 38 or claim 39, wherein the agent is selected from a WDR7 inhibitor, a CCDC115 inhibitor, and a TMEM199 inhibitor.

41. The method of any one of claims 1 to 37, wherein the agent reduces viral transcription.

42. The method of claim 41, wherein the agent is a CMTR1 inhibitor.

43. The method of any one of the preceding claims, wherein the influenza A is selected from H1N1, H3N2, H5N1, and H7N9 influenza A.

44. The method of any one of the preceding claims, wherein the influenza A is selected from HINT and H3N2 influenza A.

45. The method of any one of the preceding claims, wherein the subject is suspected of having an influenza A virus infection.

46. The method of any one of the preceding claims, wherein the subject is at risk of developing an influenza A virus infection.

47. The method of any one of the preceding claims, wherein the subject has been diagnosed with an influenza A virus infection.

48. The method of any one of the preceding claims, wherein the subject exhibits at least one symptom of influenza A virus infection.

49. The method of claim 48, wherein at least one symptom is selected from fever, muscle ache, chills, headache, cough, fatigue, nasal congestion, and sore throat.

50. The method of any one of the preceding claims, wherein treating influenza A virus infection comprises reducing the severity and/or duration of one or more symptoms of influenza A virus infection.

51. The method of any one of the preceding claims, further comprising administering a therapeutic agent selected from baloxavir marboxil, oseltamivir, and zanamivir to the subject.

52. The method of any one of the preceding claims, wherein the method comprises administering a CMTR1 inhibitor and baloxavir marboxil to the subject.
